(19) United States
(12) Patent Application Publication
(10) Pub. No.: US 2013/0274892 A1
(43) Pub. Date: Oct. 17, 2013

(54) Electrospun Mineralized Chitosan Nanofibers Crosslinked with Genipin for Bone Tissue Engineering

(71) Applicants: Peter I. Lelkes, Cherry Hill, NJ (US); Michael Frohbergh, Philadelphia, PA (US)

(72) Inventors: Peter I. Lelkes, Cherry Hill, NJ (US); Michael Frohbergh, Philadelphia, PA (US)

(73) Assignee: Drexel University, Philadelphia, PA (US)

(21) Appl. No.: 13/877,228

(22) PCT Filed: Oct. 7, 2011
(86) PCT No.: PCT/US11/55209

§ 371 Date: Jun. 10, 2013

Related U.S. Application Data
(60) Provisional application No. 61/390,918, filed on Oct. 7, 2010.

(51) Int. Cl. A61F 2/28

(52) U.S. Cl. CPC A61F 2/28
USPC 623/23.63; 623/23.61

(57) ABSTRACT

The present invention relates to an electrospun chitosan scaffold. The scaffold mimics natural bone mechanical properties and structure through mineralization without hindering the biocompatibility for osteoblasts. The electrospun mineralized scaffold is useful in bone regeneration therapies.

### CROSS-REFERENCE TO RELATED APPLICATIONS

The present application is entitled to priority under 35 U.S.C. §119(e) of U.S. Provisional Application No. 61/390,918, filed on Oct. 7, 2010, which application is hereby incorporated by reference in its entirety.

### BACKGROUND OF THE INVENTION

Reconstruction of large bone defects formed as a result of trauma, resection, or congenital malformations remains problematic in orthopedic and craniofacial clinical practice. With the goal of shifting the strategy from prosthetic replacement to regeneration, bone tissue engineering using osteo-inductive and -conductive scaffolds may offer an alternative approach to overcome this problem.

A variety of methods, including autologous grafts, allografts, and alloplastic materials are used for treatment of bone defects. The use of bone grafts is conventional practice in orthopedics. Common orthopedic uses of bone grafts INCLUDE the management of non-unions and acute long bone fracture, joint reconstruction and to facilitate fusion of vertebral motion segments in treating a variety of spinal disorders (Lane, 1987, Ortho Clin N Amer 18; 213-225).

Currently, the most clinically acceptable grafting material is autologous bone. So-called autografts are often obtained from a secondary operative site. There are significant issues associated with autografts. These include lack of an adequate supply for large wounds or defects. Elderly individuals with osteoporosis or osteopenia make the use of an autograft problematic. The secondary morbidity associated with the harvesting operation is high. These complications include infections, pelvic instability (the bone is often harvested from the iliac crest), hematoma, and pelvic fracture (Laurie et al., 1984, Plas Rec Surg 73:933-938; Summers et al. (1989) J Bone Joint Surg 7113; 677.680; Younger et al., 1989, J Orthop Trauma, 3:192-195; Kurz at al., 1989, Spine 14; 1324-1331). In addition, chronic pain at the donor site is the second most frequently reported complication (Turner et al., 1992, JAMA 268; 907-911). Finally, the ability to shape the autograft to the defect/wound site is limited due to the rigid nature of the material. Other grafting material, including allografts and prosthetic materials are associated with poor integration with host bone, fragmentation, and displacement (Borden et al, 2002, Biomaterials, 23(2); 551-9; Ransford et al., 1998, J Bone Joint Surg Br, 80(1): 13-8).

Bone is a unique triphasic tissue that contains cellular components, hydrated extracellular organic matrix, and an extracellular mineral phase, mainly composed of calcium phosphate in the form of hydroxyapatite (HA) (Veis, 1988, Ciba Found Symp, 161:77; Shi et al., 1996, J Bone Miner Res, 11(8): 1139-45; Boskey et al., 1984, Orthop Clin North Am, 15(4): 597-612). The outer layer, the periosteum, contains multipotent mesenchymal stem cells and osteoprogenitor cells that contribute to growth and regeneration of bone (Allen et al, 2004, Bone, 35(5): 1003-12). It has been reported that mechanical properties at the graft-host tissue junction remain impaired in comparison to autografts due to the lack of new bone formation around this junction (Zhang 2008, Clin Orthop Relat Res, 466(8): 1777-87).

Since an optimal bone substitute still remains to be identified, a wide array of fabrication techniques and materials has been proposed to create biomimetic scaffolds that would aid in bone regeneration. Recent investigations have focused on the use of a variety of matrices, either inorganic such as hydroxyapatite (Flatley et al., 1983, Clin Orthop Rel Res 179:246-252; Shima et al., 1979, J Neurosurg 51:533-538; Whitehill et al., 1985, Spine 10:32-41; Herron, et al., 1989, Spine 14:496-500; Cook et al., 1986, Spine 11; 305-309; the contents of which are incorporated herein by reference) or organic such as demineralized bone matrix (DBM) (reviewed in Ashay et al., 1995, Am J Orthop 24:752-761; the contents of which are incorporated herein by reference). These matrices are thought to be osteoconductive (facilitate the invasion of bone forming cells in an inert matrix) or osteoinductive (induce the transformation of recruited precursor cells to osteoblasts). A number of successful clinical outcomes have been observed with some of these products approved for use clinically by the Food and Drug Administration. In spite of these successes, a number of issues remain for the utility of these matrices. The first is the variable subject response to DBM. Also these matrices take much longer than autologous bone transplantation to develop significant structural integrity and bear load effectively.

An alternative to transplantation and the use of simple matrices is the admixture of progenitor cells (e.g., stem cells, bone marrow stromal cells, and the like) with DBM. Ideally the cells and DBM will be derived from the same subject although allogeneic DBM has already been used clinically with initial success (Mulliken et al., 1981, Ann Surg 194:366-372; Kaban et al., 1982, J Oral Maxillofac Surg 40:623-626). Transplantation methods using autologous cells with allogeneic DBM have yielded good results (Connolly, 1995, Clin Orthop 313:8-18).

In recent years, the technology of electrospinning has allowed scientists to produce nanofibrous scaffolds, which are superior in surface area and biomimetic properties (Cai et al., 2010, J Biomed Mater Res A, 95(1): 49-57). Nanofibrous architecture may be beneficial in terms of the proliferation, differentiation, and mineralization of osteoprogenitor cells (Woo et al., 2007, Biomaterials, 28(2): 335-43). Co-electrospinning, i.e. combing polymers with bioactive substances such as HA, can further improve the biomimetic properties of nanofibrous scaffolds and enhance cell attachment, osteoblastic differentiation, and bone extracellular cell matrix (ECM) synthesis (Ito et al., 2005, J Biosci Bioeng, 100(1): 43-9; Kim et al., 2006, J Biomed Mater Res A, 79(3): 643-9; Venugopal et al., 2008, J Mater Sci Mater Med, 19(5): 2039-46). Chitosan (CTS) (Zhang et al., 2008, Biomacromolecules, 9(1): 136-41; Pramanik et al., 2009, Int J Biomat, 512417; Bhattari et al., 2005, Biomaterials, 26(31): 6176-84), the deacetylated form of chitin, a protein derived from the exoskeleton of crustaceans (Prasitslip et al., 2000, J Mater Sci Mater Med, 11(12): 773-8) has emerged as a promising candidate for bone tissue engineering, mainly due to its biocompatibility and structural similarity to bone ECM (Zhang et al., 2008, Biomaterials, 29(32): 4314-22; Kumirska et al., 2010, Mar Drugs, 8(5): 1567-636). Recent approaches focus on co-electrospinning CTS with other materials, such as collagen, poly(lactic-co-glycolic acid) (PLGA) and poly(caprolactone) (PCL) for various applications (Venugopal et al., 2008, J Mater Sci Mater Med, 19(5): 2039-46; Zhang et al., 2010, Tissue Eng Part A, 16(6): 1949-60; Thomas et al., 2007, Biomacromolecules, 8(2); 631-7; Teng et al., 2008, J Mater Sci Mater Med, 19(6): 2453-61; Chen et al., 2011, Int J Biol Macromol, 48(1); 13-9; Xie et al., 2010, Biomed Mater, 5(6): 065016). However, little attention has been paid to generating fibrous chitosan based scaffolds which mechanically and physically mimic the periosteum, the layer of bone that is responsible for the success of autografts over allografts and engineered constructs.

Despite advances in tissue engineering, there is no ideal solution and natural bone-like scaffold with the appropriate mechanical properties of bone and osteoblast biocompatibility. Accordingly, there remains a need for a model for mineralized nanofibrous scaffolds for bone tissue engineering. The present invention fulfills this need.

### BRIEF SUMMARY OF THE INVENTION

The invention provides a scaffold comprising an electroprocessed cross-linked mineralized chitosan nanofiber, wherein the scaffold is substantially free of a fiber forming agent and wherein the scaffold is capable of supporting the maturation of an osteoblast.

In one embodiment, the mineralized chitosan nanofiber comprises hydroxyapatite.

In one embodiment, the mineralized chitosan fiber is cross-linked with genipin.

In one embodiment, the fiber forming agent is polyethylene oxide.

In one embodiment, the scaffold exhibits mechanical properties of natural bone.

In one embodiment, the scaffold further comprises a cell selected from the group consisting of an osteoprogenitor cell, a mesenchymal cell, a stein cell, an osteoblast, an osteocyte, and any combination thereof.

The present invention also provides a method of making a scaffold comprising an electroprocessed cross-linked mineralized chitosan nanofiber. In one embodiment, the method comprises: (a) dissolving chitosan and hydroxyapatite in an acidic solution to form a first solution, (b) electroprocessing the first solution to form a fibrous mat, and (c) cross-linking the fibrous mat with genipin to form a scaffold comprising an electroprocessed cross-linked mineralized chitosan nanofiber.

In one embodiment, the acidic solution comprises trifluoracetic acid.

In one embodiment, the method further comprises contacting a cell with the scaffold, wherein the cell is selected from the group consisting of an osteoprogenitor cell, a mesenchymal cell, a stem cell, an osteoblast, an osteocyte, and any combination thereof.

The invention also provides a method of treating a bone defect in a mammal. In one embodiment, the method comprises administrating to the site of a bone defect a scaffold comprising an electroprocessed cross-linked mineralized chitosan nanofiber, wherein the scaffold is substantially free of a fiber forming agent and wherein the scaffold is capable of supporting the maturation of an osteoblast.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a photograph and schematic illustrating the electrospinning set up. The electrospinning solution is extruded from the syringe via an automatic syringe pump (a). An electrical field is generated by a high voltage power supply between the cathode connected to the needle tip (c) and the anode (d) connected to the collecting plate (b).

FIG. 2, comprising FIGS. 2A and 2B, are images of a representative electrospun non-mineralized chitosan scaffold macroscopically (FIG. 2A) and microscopically (FIG. 2B). Scale bar for FIG. 2A is 1 cm and for FIG. 2B is 200 μm.

FIG. 3, comprising FIGS. 3A through 3C, are images showing the morphology of electrospun scaffolds. FIG. 3A shows a scanning electron micrograph of 0.1% genipin crosslinked non-mineralized 7% chitosan nanofibers, while FIG. 3B shows a scanning electron micrograph of 0.1% genipin crosslinked 1% mineralized 7% chitosan nanofibers. Scale bars are 200 nm. Inserts: Typical morphologies of nanofibers at lower magnification (scale bar for insert in FIG. 3A is 2 μm and in FIG. 3B is 1 μm).

FIG. 3C shows atomic force topography of 7% chitosan nanofibers mineralized with 1% HA, crosslinked with genipin. Topographical representation of the fibrous structure illustrated by arrows, scale bar is 200 nm.

FIG. 4, comprising FIGS. 4A through 4C, is a series of images depicting X-ray diffraction spectra of hydroxyapatite (FIG. 4A), 0.1% genipin crosslinked 7% chitosan nanofibers (FIG. 4B) and 1.0% mineralized 0.1% genipin crosslinked 7% chitosan nanofibers (FIG. 4C). The alignment of peaks is indicated by dashed lines.

FIG. 5, comprising FIGS. 5A through 5C, is a series of images depicting fonder transform infrared spectra of hydroxyapatite (FIG. 5A), 0.1% genipin crosslinked 7% chitosan nanofibers (FIG. 5B) and 1.0% mineralized 0.1% genipin crosslinked 7% chitosan nanofibers (FIG. 5C). Areas of interest are marked by arrows.

FIG. 6, comprising FIGS. 6A through 6E, depicts electron dispersion spectroscopy of non-mineralized and mineralized 7% chitosan nanofibers. FIG. 6A is an image showing the spectral analyses comparing the elemental compositions of non-mineralized and 1.0% mineralized (insert) 0.1% genipin crosslinked 7% chitosan nanofibers. Also shown are dot-analyses representing the elemental topographical distribution of carbon (FIG. 6B), oxygen (FIG. 6C), calcium (FIG. 6D) and phosphorous (FIG. 6E) of the above mineralized nanofibers.

FIG. 7 is an image depicting Von Kossa staining of 0.1% genipin crosslinked 1.0% mineralized 7% chitosan nanofibers. Insert: Control non-mineralized nanofibers. The von Kossa stain shows micro size calcium deposits on the surface of the scaffold, which indicates that some HA is not being fully incorporated onto the fibers and is rather being sprayed onto the surface of the scaffold during the electrospinning process.

FIG. 8, comprising panels 8A and 8B, is an image depicting the ultimate tensile strength (FIG. 8A), as measured by the stress at break as a function of the cross-sectional area of the scaffold, and Young's modulus (FIG. 8B) of non-crosslinked (white bars) 0.1% genipin crosslinked (black bars) 7% chitosan nanofibers at different concentrations of hydroxyapatite. ** indicates statistical significance at p<0.01 with one way ANOVA with Tukey test.

FIG. 9 is an image depicting growth curves of 7F2 osteoblasts on different conditions of 0.1% genipin crosslinked non-mineralized and 1.0% mineralized 7% chitosan nanofibers and various coated chitosan nanofibers. Cell growth was monitored with alamar blue for up to one week. Bis-benzamide and phalloidin staining (right) stain for nuclei and actin cytoskeleton respectively, indicating cell spreading after 3 days.

FIG. 10, comprising FIGS. 10A through 10L are SEM micrographs of 7F2 cells on non-mineralized 0.1% genipin crosslinked chitosan nanofibers at day 7 (FIGS. 10A and 10B), day 14 (FIGS. 10E and 10F), and day 21 (FIGS. 10I and 10J) and 1.0% mineralized 0.1% genipin crosslinked chitosan nanofibers at day 7 (FIGS. 10C and 10D), day 14 (FIGS. 10G and 10H), and day 21 (FIGS. 10K and 10L).

FIG. 11, comprising FIGS. 11A through 11D, are images depicting metabolic activity, alkaline phosphatase expression and mRNA expression of 7F2 osteoblasts. Images show alkaline phosphatase expression of 7F2 osteoblasts on days 1, 7, 14, and 21 (FIG. 11A) and metabolic activity of 7F2 osteoblasts on days 1, 3, 7, 14, and 21 (FIG. 11B). Images also show expression of osteopontin and osteonectin mRNA of 7F2 osteoblasts on days 1, 14 and 21 on non-mineralized (FIG. 11C) and mineralized (FIG. 11D) 0.1% genipin crosslinked chitosan nanofibers.

### DETAILED DESCRIPTION OF THE INVENTION

The present invention relates to an electrospun chitosan scaffold. The scaffold mimics natural bone mechanical properties and structure through mineralization without hindering the biocompatibility for bone cells. The electrospun mineralized scaffold is useful in bone regeneration therapies.

The present invention is based on the successful fabrication of a biomimetic scaffold useful for bone tissue engineering. In one embodiment, the scaffold is a mineralized biomimetic scaffold that can be generated in a one-step electrospinning procedure using an acidic solution containing hydroxyapatite nanoparticles. In some instances, it is preferable that the electrospun material is crosslinked with a natural crosslinking agent, such as genipin, in order to increase the mechanical properties of the scaffold.

In one embodiment, the scaffold is fabricated from an acidic solution comprising a natural biomacromolecule, preferably chitosan. This solution can be supplemented with a suspension of hydroxyapatite, preferably nanosized hydroxyapatite. Under optomized spinning conditions, the outcome results in a homogenous submicron sized, mineralized chitosan fibers, with no need for fiber-forming agents, such as PEO, or postfabrication mineralization procedures. Crosslinking with a natural crosslinking agent, such as genipin, further stabilizes the scaffolds and increases the mechanical strength thereby making the scaffold of the present invention a “natural” bioactive scaffold suitable for regenerative therapy.

In one embodiment, the mechanical properties of the scaffold of the present invention is comparable to that of periosteum. For example, the scaffold of the invention is structurally similar to bone and exhibits the tensile properties of periosteum. The scaffold also supports the bioactivity of bone. Under optomized conditions, the scaffold supports the differentiation, proliferation, and maturation of osteoblasts. The scaffold is osteogenic and is an effective substrate for bone growth and formation.

The invention also provides a method of alleviating or treating a bone defect in a mammal, preferably a human. The method comprises administering to the mammal in need thereof a therapeutically effective amount of a composition comprising a chitosan scaffold of the invention, thereby alleviating or treating the bond defect in the mammal. The method can further comprise culturing cells on the scaffold of the invention, ex vivo, prior to administration of the composition. The cells used in this method can include osteoprogenitor cells, mesenchymal cells, stem cells, osteoblasts, osteocytes, and the like.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well-known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Laboratory Press, Cold Spring Harbor, N.Y., and Ausubel et al., 2002, Current Protocols in Molecular Biology, John Wiley & Sons, New York, N.Y.), which are provided throughout this document.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent based on the context in which it is used.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, the term “bone condition (or injury or disease)” refers to disorders or diseases of the bone including, but not limited to, acute, chronic, metabolic and non-metabolic conditions of the bone. The term encompasses conditions caused by disease, trauma or failure of the tissue to develop normally. Examples of bone conditions include, but are not limited, a bone fracture, a bone/spinal deformation, osteosarcoma, myeloma, bone dysplasia, scoliosis, osteoporosis, osteomalacia, rickets, fibrous osteitis, renal bone dystrophy, and Paget's disease of bone.

“Differentiation medium” is used herein to refer to a cell growth medium comprising au additive or a lack of an additive such that a stem cell or progenitor cell, that is not fully differentiated, develops into a cell with some or all of the characteristics of a differentiated cell when incubated in the medium.

The term “electroprocessing” as used herein shall be defined broadly to include all methods of electrospinning, electrospraying, electroaerosoling, and electrosputtering of materials, combinations of two or more such methods, and any other method wherein materials are streamed, sprayed, sputtered or dripped across an electric field and toward a target. The electroprocessed material can be electroprocessed from one or more grounded reservoirs in the direction of a charged substrate or from charged reservoirs toward a grounded target. “Electrospinning” means a process in which fibers are formed from a solution or melt by streaming an electrically charged solution or melt through an orifice. “Electroaerosoling” means a process in which droplets are formed from a solution or melt by streaming an electrically charged polymer solution or melt through an orifice. The term electroprocessing is not limited to the specific examples set forth herein, and it includes any means of using an electrical field for depositing a material on a target.

The terms “hydroxylapatite”, “hydroxyapatite” and “HA” are used interchangeably to refer to a mineral that is the major constituent of bone and tooth mineral. It is a finely divided, crystalline, nonstoichiometric material rich in surface ions (including carbonate, magnesium, and citrate ions). It is thermodynamically stable at physiological pH (meaning it does not break down under physiological conditions) and may form strong chemical bonds with surrounding bone.

As used herein, the term “solution” is used to describe the liquid in the reservoirs of the electroprocessing method. The term is defined broadly to include any liquids that contain materials to be electroprocessed. It is to be understood that any solutions capable of forming a material during electroprocessing are included within the scope of the present invention. “Solutions” can be in organic or biologically compatible forms. This broad definition is appropriate in view of the large number of solvents or other liquids (polar and non-polar) and carrier molecules that can be used in the many variations of electroprocessing.

As used herein, a “substantially purified” cell is a cell that is essentially free of other cell types. Thus, a substantially purified cell refers to a cell which has been purified from other cell types with which it is normally associated in its naturally-occurring state.

“Expandability” is used herein to refer to the capacity of a cell to proliferate, for example, to expand in number or, in the case of a population of cells, to undergo population doublings.

As used herein, a “graft” refers to a cell, tissue, organ, scaffold, and the like that is implanted into an individual, typically to replace, correct or otherwise overcome a defect. The graft may comprise of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft”, “allogeneic transplant”, “allogeneic implant” and “allogeneic graft”. A graft from an individual to his identical twin is referred to herein as an “isograft”, a “syngeneic transplant”, a “syngeneic implant” or a “syngeneic graft”. A “xenograft”, “xenogeneic transplant” or “xenogeneic implant” refers to a graft from one individual to another of a different species.

As used herein, the term “growth medium” is meant to refer to a culture medium that promotes growth of cells. A growth medium will generally contain animal serum. In some instances, the growth medium may not contain animal serum.

As used herein, the term “growth factor product” refers to a protein, peptide, mitogen, or other molecule having a growth, proliferative, differentiative, or trophic effect on a cell. Growth factors include, but are not limited to, fibroblast growth factor (FGF), basic fibroblast growth factor (bFGF), acidic fibroblast growth factor (aFGF), epidermal growth factor (EGF), insulin-like growth factor-I (IGF-T), growth factor-II (IGF-II), platelet-derived growth factor (PDGF), vascular endothelial cell growth factor (VEGF), activin-A, bone morphogenic proteins (BMPs), insulin, growth hormone, erythropoietin, thrombopoietin, interleukin 3 (IL-3), interleukin 6 (IL-6), interleukin 7 (IL-7), macrophage colony stimulating factor, c-kit ligand/stem cell factor, osteoprotegerin ligand, insulin, nerve growth factor, ciliary neurotrophic factor, cytokines, chemokines, morphogens, neutralizing antibodies, other proteins, and small molecules. Preferably, the FGF is selected from the group selected from FGF2, FGF7, FGF10, and any combination thereof.

An “isolated cell” refers to a cell which has been separated from other components and/or cells which naturally accompany the isolated cell in a tissue or mammal.

As used herein, the terms “mesenchymal cells,” “mesenchymal stem cells (MSC),” or “marrow stromal cells” are used to refer to multipotent stem cells that differentiate from CFU-F cells capable of differentiating along several lineage pathways into osteoblasts, chondrocytes, myocytes and adipocytes.

The term “osteoinduction” or “osteoinductive” as used herein refers to a material having the ability to stimulate stem cells to differentiate into mature cells.

The term “osteogenesis” as used herein refers to a material having the ability to produce new bone.

As used herein, “osteogenic” refers to a material that induces the development of some or all of the characteristics of an osteoblast or osteocyte in a stem cell, adipose-derived adult stein cell or other such progenitor cell that is not fully differentiated.

As used herein, “osteogenic medium” refers to a differentiation medium that induces development of some or all of the characteristics of an osteoblast or osteocyte.

As used herein, an “osteogenic stimulant” refers to an additive that is capable of inducing some or all of the characteristics of an osteoblast or osteocyte in a stem cell, adipose-derived adult stem cell or other such progenitor cell that is not fully differentiated.

The term “osteoblasts” as used herein refers to cells that arise when osteoprogenitor cells or mesenchymal cells, which are located near all bony surfaces and within the bone marrow, differentiate under the influence of growth factors. Osteoblasts, which are responsible for bone matrix synthesis, secrete a collagen rich ground substance essential for later mineralization of hydroxyapatite and other crystals. The collagen strands to form osteoids: spiral fibers of bone matrix. Osteoblasts cause calcium salts and phosphorus to precipitate from the blood, which bond with the newly formed osteoid to mineralize the bone tissue. Once osteoblasts become trapped in the matrix they secrete, they become osteocytes. In some instances, from least to terminally differentiated, the osteocyte lineage is (i) colony-forming unit-fibroblast (CFU-F); (ii) mesenchymal stem cell/marrow stromal cell (MSC); (iii) osteoblast; (iv) osteocyte. The term “osteogenesis” refers to the formation of new bone from bone forming or osteocompetent cells.

The term “patient” as used herein includes human and veterinary subjects.

The terms “poly(glycolic acid)”, polyglycolide, and “PGA” are used interchangeably herein to refer to a biodegradable, thermoplastic polymer and the simplest linear, aliphatic polyester. PGA may be obtained commercially, for example, from Sigma-Aldrich.

A “polylactide” is a biodegradable polymer derived from lactic acid. Poly(lactide) or PLA exists in two stereo forms, signified by a D or L for dexorotary or levorotary, or by DL for the racemic mix. The term “PLLA” refers to the biodegradable aliphatic polyester homopolymer poly L-lactic acid. PLLA may be obtained commercially, for example, from Alkermes, Inc.

The terms poly(lactic acid-glycolic acid), poly(D,L-lactide-c-glycoside), and PLGA are used interchangeably to refer to a copolymer of polylactic acid and glycolic acid. PLGA may be obtained commercially, for example, from Alkermes, Inc.

The terms “precursor cell,” “progenitor cell,” and “stem cell” are used interchangeably in the art and as used herein refer either to a pluripotent or lineage-uncommitted progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like.

As used herein, “scaffold” refers to a structure, comprising a biocompatible material that provides a surface suitable for adherence, proliferation, and differentiation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, the term “stem cells” refers to undifferentiated cells having high proliferative potential with the ability to self-renew that may migrate to areas of injury and may generate daughter cells that may undergo terminal differentiation into more than one distinct cell phenotype. These cells have the ability to differentiate into various cells types and thus promote the regeneration or repair of a diseased or damaged tissue of interest. The term “cellular differentiation” refers to the process by which cells acquire a cell type. The term “progenitor cell” as used herein refers to an immature cell in the bone marrow that may be isolated by growing suspensions of marrow cells in culture dishes with added growth factors. Progenitor cells are referred to as colony-forming units (CFU) or colony-forming cells (CFC). The specific lineage of a progenitor cell is indicated by a suffix, such as, but not limited to, CFU-F

As used herein, “tissue engineering” refers to the process of generating tissues ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein, to “treat” means reducing the frequency with which symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered.

“Tissue cell culture” as used herein refers to an aggregation of cells and intercellular matter performing one or more functions in an organism. Examples of tissues include, but are not limited to, epithelium, connective tissues (e.g., bone, blood, cartilage), muscle tissue and nerve tissue.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5, 3, and 6. This applies regardless of the breadth of the range.

### DESCRIPTION

The present invention provides an electrospun chitosan scaffold. The scaffold mimics natural bone mechanical properties and structure through mineralization without hindering the biocompatibility for osteoblasts. The electrospun mineralized scaffold is useful in bone regeneration therapies.

In one embodiment, the electrospun chitosan scaffold is generated by crosslinking the chitosan scaffold with about 0.1% genipin. Preferably, the electrospun chitosan has a fiber diameter of about 50-500 nm.

In another embodiment, the crosslinked chitosan fibers are incorporated with about 1.0% hydroxyapatite (HA) in order to generate a more compatible scaffold that mimics the mineralized nature of the bone. The incorporation of HA to the crosslinked chitosan fibers also generates a higher Young's modulus of up to about 150 MPa and higher tensile strength of up to about 60 MPa.

In one embodiment, the scaffold comprises chitosan, HA, and genipin. In a preferred embodiment, the scaffold comprises about 7% chitosan, about 1% HA, and about 1% genipin. In one embodiment, the scaffold does not comprise a fiber-forming agent or a fiber-forming aid.

In one embodiment, the scaffold of the invention exhibits properties of natural bone and periosteum which allows the scaffold to function as both osteoconductive and osteoinductive material. In one embodiment, the scaffold is engineered by co-electrospinning chitosan with HA to make it structurally similar to bone and crosslinking with genipin to resemble the tensile properties of periosteum.

In another embodiment, the mineralization was confirmed by SEM, EDS, XRD, FTIR and Von Kossa staining. Osteoblasts adhere, proliferate, and differentiate on the scaffold demonstrating osteogenic properties of the invention. As described herein, electrospun chitosan nanofibers can be induced to closely mimic natural bone mechanical properties and structure through mineralization without hindering the biocompatibility for osteoblasts. Thus, the invention provides a suitable substrate for the ex vivo or in vivo regeneration of bone for treatment of orthopedic disorders.

### The Fabrication of Tissue Engineering Scaffolds

Electrospinning is a fiber forming technique that relies on charge separation to produce nano- to microscale fibers, which typically form a non-woven matrix. The terms “nonwoven matrix”, “nonwoven mesh” or “nonwoven scaffold” are used interchangeably herein to refer to a material comprising a randomly interlaced fibrous web of fibers. Generally, individual electrospun fibers have large surface-to-volume and high aspect ratios resulting from the smallness of their diameters. These beneficial properties of the individual fibers are further enhanced by the porous structure of the non-woven fabric, which allows for cell infiltration, cell aggregation, and tissue formation.

The electrospinning process is affected by varying the electric potential, flow rate, solution concentration, capillary-collector distance, diameter of the needle, and ambient parameters like temperature. Therefore, it is possible to manipulate the porosity, surface area, fineness and uniformity, diameter of fibers, and the pattern thickness of the matrix.

Electrospinning is an atomization process of a fluid which exploits the interactions between an electrostatic field and the fluid. That is, electrospinning is a method of electrostatic extrusion used to produce sub-micron sized fibers. In one aspect, the fluid can be a conducting fluid. Also known within the fiber forming industry as electrostatic spinning, the process of electrospinning generally involves the creation of an electrical field at the surface of a liquid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The resulting electrical forces create a jet of liquid which carries electrical charge. Thus, the liquid jets may be attracted to other electrically charged objects at a suitable electrical potential. As the jet of liquid elongates and travels, it will harden and dry. Fibrils of nanometer-range diameter can be produced. The hardening and drying of the elongated jet of liquid may be caused by cooling of the liquid, by evaporation of a solvent, or by a curing mechanism. The produced fibers are collected on a suitably located, oppositely charged receiver and subsequently removed from it as needed, or directly applied to an oppositely charged generalized target area.

Fibers can be electrospun from high viscosity polymer melts or polymers dissolved in volatile solvents; the end result is a non-woven mesh of fiber. Solution viscosity can be controlled by modifying polymer concentration, molecular weight, and solvents. Electric field properties can be controlled by modifying bias magnitude or tip-to-target distance. Polymers can be co-spun from same the solution and the polymer phase can be selectively removed. Further, fibers can be electrospun from a multiphasic polymer solution or from an emulsion. For example, polyurethane fibers can be electrospun from a multiphasic polyurethane solution. Emulsifying the solution can increase the solution viscosity, thereby inducing fiber formation at lower concentrations. The resultant fibers can be created having diameters as a function of aqueous content.

Electrospinning is an attractive process for fabricating scaffolds for tissue engineering applications due to the simplicity of the process and the ability to generate microscale and nanoscale features with synthetic and natural polymers. To date, a broad range of polymers has been processed by electrospinning, including polyamides, polylactides, cellulose derivatives, water soluble polymers such as polyethyleneoxide, as well as polymer blends or polymers containing solid nanoparticles or functional small molecules. To date, electrospun fibrous scaffolds have been fabricated with numerous synthetic biodegradable polymers, such as poly(ε-caprolactone) (PCL), poly(lactic acid) (PLA), poly(glycolic acid) (PGA), and the copolymers poly(lactide-co-glycolide) (PLGA). Electrospun scaffolds have been proposed for use in the engineering of bone tissue and cardiac grafts. Similarly, poly(L-lactide-co-ε-caprolactone) [P(LLA-CL)] has been electrospun into nanofibrous scaffolds for engineering blood vessel substitutes. However, the invention also includes the use of degradable polymers.

In the most fundamental sense, the electrospinning apparatus for electrospinning material includes an electrodepositing mechanism and a target substrate. The electrodepositing mechanism includes a reservoir or reservoirs to hold the one or more solutions that are to be electrospun or electrodeposited. The reservoir or reservoirs have at least one orifice or nozzle to allow the streaming of the solution from the reservoirs. One or a plurality of nozzles may be configured in an electrospinning apparatus. If there are multiple nozzles, each nozzle is attached to one or more reservoirs containing the same or different solutions. Similarly, there can be a single nozzle that is connected to multiple reservoirs containing the same or different solutions. Multiple nozzles may be connected to a single reservoir. Because different embodiments involve single or multiple nozzles and/or reservoirs, any references herein to one or nozzles or reservoirs should be considered as referring to embodiments involving single nozzles, reservoirs, and related equipment as well as embodiments involving plural nozzles, reservoirs, and related equipment. The size of the nozzles can be varied to provide for increased or decreased flow of solutions out of the nozzles. One or more pumps used in connection with the reservoirs can be used to control the flow of solution streaming from the reservoir through the nozzle or nozzles. The pump can be programmed to increase or decrease the flow at different points during electrospinning.

The electrospinning occurs due to the presence of a charge in either the orifices or the target, while the other is grounded. In some embodiments, the nozzle or orifice is charged and the target is shown to be grounded. Those of skill in the electrospinning arts will recognize that the nozzle and solution can be grounded and the target can be electrically charged. The creation of the electrical field and the effect of the electrical field on the electroprocessed materials or substances aid in the formation of the electroprocessed composition.

Any solvent can be used that allows delivery of the material or substance to the orifice, tip of a syringe, or other site from which the material will be electroprocessed. The solvent may be used for dissolving or suspending the material or the substance to be electroprocessed. Solvents useful for dissolving or suspending a material or a substance depend on the material or substance. Electrospinning techniques often require more specific solvent conditions. For example, polyurethane can be electrospun as a solution or suspension in water, 2,2,2-trifluoroethanol, 1,1,1,3,3,3-hexafluoro-2-propanol (also known as hexafluoroisopropanol or HFIP), or combinations thereof. Alternatively, polyurethane can be electrospun from solvents such as urea, monochloroacetic acid, water, 2,2,2-trifluoroethanol, HFIP, or combinations thereof. Other lower order alcohols, especially halogenated alcohols, may be used. Additional solvents that may be used or combined with other solvents include acetamide, N-methylformamide, N,N-dimethylformamide (DMF), dimethylsulfoxide (DMSO), dimethylacetamide, N-methylpyrrolidone (NMP), acetic acid, trifluoroacetic acid, ethyl acetate, acetonitrile, trifluoroacetic anhydride, 1,1,1-trifluoroacetone, maleic acid, hexafluoroacetone.

In general, when producing fibers using electrospinning techniques, it is preferable to use the monomer of the polymer fiber to be formed. In some embodiments it is desirable to use monomers to produce finer filaments. In other embodiments, it is desirable to include partial fibers to add material strength to the matrix and to provide additional sites for incorporating substances.

In addition to the multiple equipment variations and modifications that can be made to obtain desired results, similarly the electrospun solution can be varied to obtain different results. For instance, any solvent or liquid in which the material is dissolved, suspended, or otherwise combined without deleterious effect on the process or the safe use of the matrix can be used. Materials or the compounds that form materials can be mixed with other molecules, monomers or polymers to obtained the desired results. In some embodiments, polymers are added to modify the viscosity of the solution. In still a further variation, when multiple reservoirs are used, the ingredients in those reservoirs are electrosprayed separately or joined at the nozzle so that the ingredients in the various reservoirs can react with each other simultaneously with the streaming of the solution into the electric field. Also, when multiple reservoirs are used, the different ingredients in different reservoirs can be phased in temporally during the processing period. These ingredients may include other substances.

Embodiments involving alterations to the electrospun materials themselves are within the scope of the present invention. Some materials can be directly altered, for example, by altering their carbohydrate profile. Also, other materials can be attached to the matrix materials before, during or after electrospinning using known techniques such as chemical cross-linking or through specific binding. Further, the temperature and other physical properties of the process can be modified to obtain different results. The matrix may be compressed or stretched to produce novel material properties.

Electrospinning using multiple jets of different polymer solutions and/or the same solutions with different types and amounts of substances (e.g., growth factors) can be used to prepare libraries of biomaterials for rapid screening. Such libraries are desired by those in the pharmaceutical, advanced materials and catalyst industries using combinatorial synthesis techniques for the rapid preparation of large numbers (e.g., libraries) of compounds that can be screened. For example, the minimum amount of growth factor to be released and the optimal release rate from a fibrous polymer scaffold to promote the differentiation of a certain type of cell can be investigated using the compositions and methods of the present invention. Other variables include fiber diameter and fiber composition. Electrospinning permits access to an array of samples on which cells can be cultured in parallel and studied to determine selected compositions which serve as promising cell growth substrates.

One of ordinary skill in the art recognizes that changes in the concentration of materials or substances in the solutions require modification of the specific voltages to obtain the formation and streaming of droplets from the tip of a pipette.

The electrospinning process can be manipulated to meet the specific requirements for any given application of the electrospun compositions made with these methods. In one embodiment, the micropipettes can be mounted on a frame that moves in the x, y and z planes with respect to the grounded substrate. The micropipettes can be mounted around a grounded substrate, for instance a tubular mandrel. In this way, the materials or molecules that form materials streamed from the micropipettes can be specifically aimed or patterned. Although the micropipettes can be moved manually, the frame onto which the micropipettes are mounted is preferably controlled by a microprocessor and a motor that allow the pattern of streaming collagen to be predetermined by a person making a specific matrix. Such microprocessors and motors are known to one of ordinary skill in the art. For instance, matrix fibers or droplets can be oriented in a specific direction, they can be layered, or they can be programmed to be completely random and not oriented.

In the electrospinning process, the stream or streams can branch out to form fibers. The degree of branching can be varied by many factors including, but not limited to, voltage, ground geometry, distance from micropipette tip to the substrate, diameter of micropipette tip, and concentration of materials or compounds that will form the electrospun materials. As noted, not all reaction conditions and polymers may produce a true multifilament, under some conditions a single continuous filament is produced. Materials and various combinations can also be delivered to the electric field of the system by injecting the materials into the field from a device that will cause them to aerosol. This process can be varied by many factors including, but not limited to, voltage (for example ranging from about 0 to 30,000 volts), distance from micropipette tip to the substrate (for example from 0-40 cm), the relative position of the micropipette tip and target (i.e. above, below, aside etc.), and the diameter of micropipette tip (approximately 0-2 mm).

The compositions of the present invention comprise electroprocessed chitosan. In one embodiment, the scaffold comprises about 3%-15% chitosan, preferably about 5%-10% chitosan, more preferably about 7% chitosan. The invention includes chitosan electroprocessed by any means. The electroprocessed chitosan can constitute or be formed, for example, from natural chitosan, genetically engineered chitosan, or chitosan modified by conservative amino acid substitutions, non-conservative amino acid substitutions or substitutions with non-naturally occurring amino acids. The chitosan used in electroprocessing can be derived from a natural source, manufactured synthetically, or produced through any other means.

In some embodiments, the electroprocessed chitosan compositions include additional electroprocessed materials. For example, other electroprocessed materials can include natural materials, synthetic materials, or combinations thereof. Examples include, but are not limited, to amino acids, peptides, denatured peptides such as gelatin from denatured collagen, polypeptides, proteins, carbohydrates, lipids, nucleic acids, glycoproteins, minerals, lipoproteins, glycolipids, glycosaminoglycans, and proteoglycans.

Some preferred materials for electroprocessing with chitosan are naturally occurring extracellular matrix materials and blends of naturally occurring extracellular matrix materials, including, but not limited to, fibrin, elastin, laminin, fibronectin, chitin, alginates hyaluronic acid, chondroitin 4-sulfate, chondroitin 6-sulfate, dermatan sulfate, heparan sulfate, heparin, and keratan sulfate, and proteoglycans. These materials may be manufactured or isolated by any means include isolation from humans or other animals or cells or synthetically manufactured.

In some embodiments, the composition of the present invention includes additional electroprocessed materials. Other electroprocessed materials can include natural materials, synthetic materials, or combinations thereof. Some preferred examples of natural materials include, but are not limited to, amino acids, peptides, denatured peptides such as gelatin from denatured collagen, polypeptides, proteins, carbohydrates, lipids, nucleic acids, glycoproteins, lipoproteins, glycolipids, glycosaminoglycans, and proteoglycans. Some preferred synthetic matrix materials for electroprocessing with collagen include, but are not limited to, polymers such as polylactic acid) (PLA), polyglycolic acid (PGA), copolymers of PEA and PGA, polycaprolactone, poly(ethylene-co-vinyl acetate), (EVOH), poly(vinyl acetate) (PVA), polyethylene glycol (PEG) and polyethylene oxide) (PEO).

In some embodiments, the composition of the present invention comprises hydroxyapatite (HA), preferably about 0.1%-5% HA, more preferably about 0.5%-2.5% HA, most preferably about 1% HA. HA is a naturally occurring mineral form of calcium apatite and an inorganic constituent of bone and teeth. In a preferred embodiment, HA is incorporated as nanoparticles within the chitosan nanofibers. In one embodiment, the HA comprising compositions of the invention are mineralized.

The electroprocessed chitosan of the invention has the further advantage of exhibiting mechanical properties of bone and biocompatibility with bone cells. In the preferred embodiment, the scaffold of the invention displays mechanical properties similar to periosteum. In these embodiments, mechanical properties of the scaffold are influenced by cross-linking. Variation of crosslinking provides the ability to mimic natural tissue. Preferably, the electroprocessed chitosan is cross-linked with genipin. In one embodiment, the scaffold comprises about 0.1%-5% genipin, more preferably about 0.5%-2.5% genipin, most preferably about 1% genipin. Crosslinking is one of many factors that permit control of the mechanical properties of the electroprocessed matrix. Other cross-linkers that may be used with the scaffold of the invention include glutaraldehyde, epoxides (e.g., bis-oxiranes), oxidized dextran, p-azidobenzoyl hydrazide, N-[α-maleimidoacetoxy]succinimide ester, p-azidophenyl glyoxal monohydrate, bis-[β-(4-azidosalicylamido)ethyl]disulfide, bis[sulfosuccinimidyl]suberate, dithiobis[succinimidyl proprionate, disuccinimidyl suberate, 1-ethyl-3-[3-dimethylaminopropyl]carbodiimide hydrochloride (EDC), N-hydroxysuccinimide (NHS) and other cross-linking reagents known to those skilled in the art. In another embodiment utilizing a cross-linking agent, polyacrylated materials, such as ethoxylated (20) trimethylpropane triacrylate, may be used as a non-specific photo-activated cross-linking agent.

The method of making the mineralized chitosan scaffold of the invention is advantageous in that it comprises a one-step method of incorporating HA particles within chitosan nanofibers. Prior efforts in forming mineralized fibers disclose two-step processes, in which the incorporation of HA into electrospun nanofibers is achieved by first co-precipitating HA with the polymer of interest to synthesize nanocomposites and then using the nanocomposites to formulate the solution for electrospinning. In the preferred embodiment of the invention, the method of formulating mineralized chitosan nanofibers comprises the one-step method of co-dissolving chitosan and HA in TFA.

One preferred method of the instant invention has the further advantage of not comprising fiber forming agents or fiber forming aids. Such agents or forming aids are often used to enhance chain entanglement of materials that do not have high electrospinnability. As such, prior efforts to electrospin chitosan nanofibers necessitated the further addition of fiber forming agents. Non-limiting examples of fiber forming agents and aids include but are not limited to polyethylene oxide (PEO) and ultrahigh molecular weight polyethylene oxide (UHMWPEO).

Scaffolds of the invention may function as structural matrices having medical utility. For example, bone or tissue scaffolds may comprise the presently disclosed mineralized chitosan nanofibers crosslinked with genepin that optionally include one or more materials that permit, promote, or enhance biocompatibility, biological fixation, bone and/or tissue ingrowth, or other useful medical characteristics that render the bone or tissue scaffold safer, less likely to be rejected, more likely to provide structural support, more likely to be compatible with or encourage biological fixation, assimilation, and/or bone/tissue growth, among other beneficial characteristics. Many substances are incorporated into traditional bone and tissue implants, including collagen, gelatin, hydroxyapatite, etc., antiviricides, antimicrobials and/or antibiotics, amino acids, magainins, peptides, vitamins, inorganic elements, co-factors for protein synthesis, hormones, endocrine tissue or tissue fragments, synthesizers, enzymes such as collagenase, peptidases, oxidases, etc., polymer cell scaffolds with parenchymal cells, surface cell antigen eliminators, angiogenic drugs and polymeric carriers containing such drugs, biocompatible surface active agents, antigenic agents, cytoskeletal agents, cartilage fragments, living cells such as chondrocytes, bone marrow cells, mesenchymal stein cells, natural extracts, tissue transplants, bioadhesives, growth factors, growth hormones such as somatotropin, bone digestors, antitumor agents, fibronectin, cellular attractants and attachment agents, immunosuppressants, permeation enhancers, e.g., fatty acid esters such as laureate, myristate and stearate monoesters of polyethylene glycol, enamine derivatives, alpha-keto aldehydes, etc., nucleic acids; and, bioerodible polymers. These and other substances having medical utility may be included in the present scaffolds.

### Incorporation of Cells

Any cell can be used with the electroprocessed chitosan of the present invention. Thus, the invention should not be limited to the type of cell. The application of the electroprocessed chitosan in regenerative medicine is discussed in the context of MSCs merely for the sake of simplicity.

The cells of the invention may be introduced alone or in admixture with a composition useful in the repair of bone wounds and defects. MSCs can be derived in great quantities from the recipient's bone marrow and, using the novel culture conditions, can be induced to differentiate into an osteoprogenitor cell with great capacity to regenerate bone. Confined in biocompatible gels such as, but not limited to, clotted plasma, the cells build new bone at the site and induce the infiltration of new blood vessels which is a critical requirement for bone growth). The treated MSCs also produce a countless number of secreted growth factors, cytokines and structural proteins, including, but not limited to collagen I and BMP2. In the preferred embodiment of the invention, the scaffold is osteoconductive, osteoinductive, and osteogenic. Since the new material is real bone, and the cells are from the recipient or a compatible donor, rejection and maintenance is not an issue.

The present invention includes using electroprocessed chitosan to deliver cells to the desired tissue. The cells can be seeded onto or into a three-dimensional scaffold and administered in vivo in a mammal, where the seeded cells proliferate on the framework and form a replacement tissue in vivo in cooperation with the cells of the mammal.

In some aspects of the invention, the scaffold comprises extracellular matrix, cell lysate (e.g., soluble cell fractions), or combinations thereof, of the desired cells. In some embodiments, the scaffold comprises an extracellular matrix protein secreted by the cells of the invention. Alternatively, the extracellular matrix is an exogenous material selected from the group consisting of calcium alginate, agarose, fibrin, collagen, laminin, fibronectin, glycosaminoglycan, hyaluronic acid, heparin sulfate, chondroitin sulfate A, dermatan sulfate, and bone matrix gelatin. In some aspects, the matrix comprises natural or synthetic polymers.

The invention includes biocompatible scaffolds comprising electroprocessed chitosan. These scaffolds are useful in the art of cell-based therapy, surgical repair, tissue engineering, and wound healing. Preferably, the scaffold is used in treatments of orthopedic or craniofacial disorders. Non-limiting examples of conditions wherein the scaffold is useful include the reconstruction of bone defects formed as result of trauma, resection, or congenital malformations. Preferably the scaffolds are pretreated (e.g., seeded, inoculated, contacted with) with the cells, extracellular matrix, conditioned medium, cell lysate, or combination thereof. In some aspects of the invention, the cells adhere to the scaffold. In one embodiment of the invention, the scaffold is cultured with cells ex vivo. The seeded scaffold can be introduced into the mammal at any time point in the culturing process, in any way known in the art, including but not limited to implantation, injection, surgical attachment, transplantation with other tissue, injection, and the like. The scaffold can be used to fill an entire bone defect, or alternatively be used along with an autograft, allograft, or xenograft. The scaffold of the invention may be configured to the shape and/or size of a tissue or organ in vivo. For example, but not by way of limitation, the scaffold may be designed such that the scaffold structure supports the seeded cells without subsequent degradation; supports the cells from the time of seeding until the tissue transplant is remodeled by the host tissue; and allows the seeded cells to attach, proliferate, and develop into a tissue structure having sufficient mechanical integrity to support itself.

Scaffolds of the invention can be administered in combination with any one or more growth factors, cells, drugs or other and/or components described elsewhere herein that stimulate tissue formation or otherwise enhance or improve the practice of the invention. The cells to be seeded onto the scaffolds may be genetically engineered to express growth factors or drugs.

In another preferred embodiment, the cells of the invention are seeded onto a scaffold where the material exhibits specified physical properties of porosity and biomechanical strength to mimic the features of natural bone, thereby promoting stability of the final structure and access and egress of metabolites and cellular nutrients. That is, the material should provide structural support and can form a scaffold into which host vascularization and cell migration can occur. In this embodiment, the desired cells are first mixed with a carrier material before application to a scaffold. Suitable carriers include, but are not limited to, calcium alginate, agarose, types I, II, IV or other collagen isoform, fibrin, poly-lactic/poly-glycolic acid, hyaluronate derivatives, gelatin, laminin, fibronectin, starch, polysaccharides, saccharides, proteoglycans, synthetic polymers, calcium phosphate, and ceramics (i.e., hydroxyapatite, tricalcium phosphate).

The external surfaces of the three-dimensional framework may be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, and agarose.

In some embodiments, it is important to re-create in culture the cellular microenvironment found in vivo. In addition, growth factors, osteogenic inducing agents, and angiogenic factors may be added to the culture medium prior to, during, or subsequent to inoculation of the cells to trigger differentiation and tissue formation by the cells following administration into the mammal.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Electrospun Mineralized Chitosan Nanofibers Crosslinked with Genipin for Bone Tissue Engineering

Reconstruction of large bone defects remains problematic in orthopaedic and craniofacial clinical practice. While autografts, the gold standard, are limited in supply and are associated with donor site morbidity, other materials show poor integration with host bone, often due to the absence of periosteum, the layer responsible for growth and regeneration of bone. Engineering functional periosteum offers a potential solution to this problem. Described herein is a novel one-step platform technology to generate electrospun nanofibrous chitosan (CTS) scaffolds mineralized with hydroxyapatite (HA) and subsequently crosslinked with genipin to yield a microenvironment promoting osteoblast differentiation and maturation while possessing mechanical properties of periosteum. SEM revealed fiber diameters of 227±154 nm as spun and 335±119 nm after crosslinking. AFM revealed 42±11 nm HA particles with uniform distribution in the fibers. Analysis by XRD and FT-IR showed characteristic features of HA incorporated into CTS fibers. The Young's modulus of the mineralized, crosslinked fibers was 124±21 MPa, which is comparable to that of periosteum. Mouse 7F2 osteoblast-like cells adhere, proliferate and differentiate well on both mineralized and non-mineralized chitosan scaffolds. The colorimetric ALP assay revealed higher activity of this early osteogenic marker on the mineralized scaffolds compared to non-mineralized scaffolds, reaching a significant, 2.4 fold, difference by day 14 (p<0.05). Similarly, by day 14 cells cultured on HA containing CTS scaffolds had the highest rate of increase of osteonectin mRNA expression, indicating enhanced osteoinductivity of the mineralized scaffolds. Results described herein show that crosslinking electrospun CTS scaffolds with genipin enables engineering of a biocomposite combining an ECM-like biomimetic environment with mechanical properties of periosteum, which facilitates the proliferation and maturation of osteoblast-like cells.

The materials and methods employed in these experiments are now described.

### Materials and Methods

Materials

Medium molecular weight chitosan (CTS, 75%-85% deacetylated), trifluoroacetic acid (TFA, ≧98%), and hydroxyapatite (HA, reagent grade, >200 nm nanoparticles) were purchased from Sigma-Aldrich Co. Ltd. (St. Louis, Mo.). Genipin (GP, ≧98% pure) was from Wako Pure Chemical Industries Ltd. (Osaka, Japan). The alamar blue colorimetric assay kit was purchased from AbD Serotec (Raleigh, N.C.). The alkaline phosphatase calorimetric assay was purchased from Abeam (Cambridge, Mass.). All PCR kits and master mixes were purchased from Qiagen (Valencia, Calif.) and all primers from Applied Biosystems (Carlsbad, Calif.).

Electrospinning

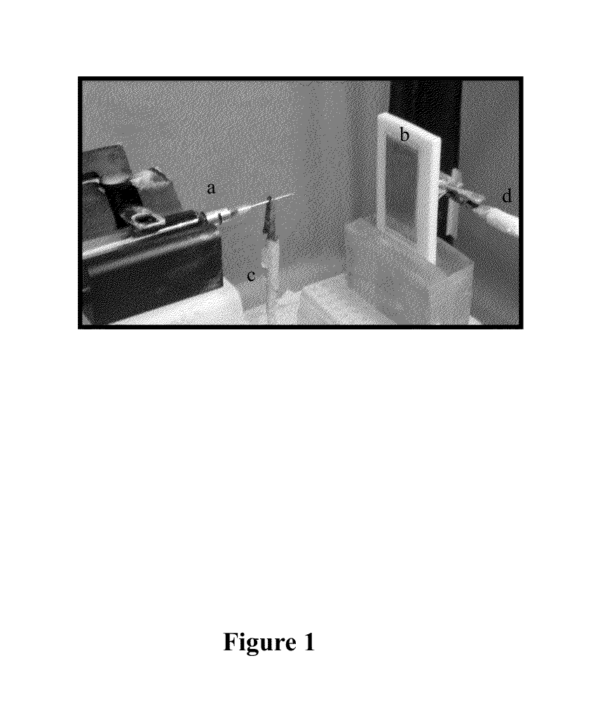

Non-mineralized scaffolds were electrospun from solution of CTS dissolved in TFA to yield 7% (w/v) CTS. Mineralized scaffolds were generated by admixing 1% HA nanoparticles (w/v) to the CTS solution. Both solutions were stirred at room temperature for at least 5 days. Electrospinning was performed in a home-made system (FIG. 1), essentially as previously described (Bhattarai et al., 2005, Biomaterials 26(31): 6179-84; Li et al., 2002, J Biomed Mater Res, 60(4): 613-21). In brief, a 5 mL glass syringe (BD, multifit syringes) containing 4 ml of the above solutions was mounted in a KDS200 syringe pump (KD Scientific), set at a flow rate of 1.2 ml/hr. A voltage of 15 kV generated by an ES-30 Gamma High Voltage Research power supply (Gamma High Voltage Research) was applied. The cathode was connected to the syringe needle and the anode was connected to the rectangular 6×2 cm aluminum collecting plate placed 15 cm from the tip of the needle.

Crosslinking

Electrospun scaffolds were crosslinked with 0.1% (w/v) GP The scaffolds were first stabilized by soaking in 0.5% sodium hydroxide (NaOH) dissolved in 100% ethanol for 20 minutes followed by five 30 seconds washes with 1× phosphate buffer solution (PBS) to remove any trace amounts of ethanol (Hsieh et al., 2007, Carbohydrate Polymers, 67(1): 124-32). The stabilized scaffolds were then crosslinked in 0.1% (w/v) genipin dissolved in 1×PBS for 24 hours. Crosslinking was stopped by washing the scaffolds as described above. The resulting scaffolds were termed chitosan-genipin crosslinked non-mineralized scaffolds (CTS-GP) or chitosan-hydroxyapatite-genipin crosslinked mineralized biocomposite scaffolds (CTS-HA-GP).

Scanning Electron Microscopy (SEM)

For ultrastructural analysis, circular scaffold samples of 10.3 mm diameter were sputter coated with carbon. The samples were viewed and digitally photographed in a Zeiss Supra50VP field emission scanning electron microscope (FESEM) equipped with an Oxford Instruments INCA Energy Dispersive Spectrometer at 5 kV with the SE2 detector using a 30 μm final aperture.

Atomic Force Microscopy (AFM)

The nanoscale topography of the scaffolds was assessed using a Veeco atomic force bioscope in conjunction with a Veeco cantilever and the Nanoscope III technology mounted on a Nikon Eclipse TE2000-U microscope in the tapping mode with a silicon tip with a spring constant k=40N/m and a tip radius of >10 nm, as previously described (Li et al., 2005, Biomaterials, 26(30): 5999-6008).

Electron Dispersion Spectroscopy (EDS)

An FESEM equipped with EDAX was used to assess calcium and phosphorous contents in the scaffolds. X-Ray spectra were taken at 10 kV using a 60 μm final aperture. EDS was performed using the FESEM at an acceleration voltage of 10 kV.

X-ray Diffraction (XRD)

XRD was performed in a Siemens D500 powder diffractometer using conventional Bragg-Brentano geometry in q-2q configuration, with Cu_{Ka} source (1-0.154 nm). 2q scans were acquired from 10-60° with a step of 0.03° and 1 s dwell time per point.

Fourier Transform Infrared Spectroscopy (FTIR)

FTIR spectra were collected on a Varian Inc FTS3000 Excalibur FTIR spectrometer equipped with a Deuterated Triglycine Sulfate (DTGS) detector and KBr beam splitter. The spectra were recorded at resolution of 4 cm⁻¹ in transmission mode.

Mechanical Properties

The mechanical properties of the scaffolds were tested using an Instron Model 5564 Table Mounted Materials Testing System and Merlin IX software. Hydrated CTS-GP and CTS-HA-GP scaffolds were cut into strips of 22.7±2.3 mm×5.4±0.7 mm×0.02±008 mm. A gauge length of 10 mm was used for all samples. The stretching speed was 1 mm/min. The thickness of the scaffolds was determined by using equation 1 seen below.

$$T = \frac{m}{l \times w \times p} \qquad ( l )$$

where T is the thickness of the strip, in is the mass of the strip (weighed on Mettler Toledo ab54-S balance), l is the length of the strip, is the width of the strip and p is the specific density of chitosan (1.45 g/cm³) (UChitotech Specifications). Average thickness of the scaffolds was 25.3±16.2 μm.

Cell Culture

Murine 7F2 osteoblast-like cells obtained from ATCC were cultured in alpha modification of Minimum Essential Medium (α-MEM) containing 1 g/L glucose, 10% fetal bovine serum (FBS), 2 mM L-glutamine, 1% (v/v) penicillin-streptomycin in T-cell culture flasks in an incubator set to 37° C., 5% carbon dioxide. The medium was changed every second day. The cells were passaged three times by gentle trypsinization prior to seeding onto the scaffolds, as described elsewhere herein.

Seeding of 7F2 Cells on Scaffolds

Circular samples with a diameter of 10.3 mm, cut from the CTS-HA-GP (1.0% HA) and CTS-GP scaffolds, were placed in 24 well plates, scoured with a VitonO-ring, stabilized, and crosslinked with 0.1% GP, as described above. The samples were sterilized with UV light for one hour and pre-treated by soaking with complete medium overnight, 7F2 cells were seeded in aliquots of 50 μl containing 10,000 cells by carefully pipetting onto the center of the scaffold and placed in an incubator for one hour. After this period, 450 μL medium consisting of low glucose (1 g/L) α-MEM, 10% FBS, 2 mM L-glutamine, 1% (v/v) penicillin-streptomycin, was added to each well. The cells were cultured for up to 21 days during which the medium was changed every other day. Cells were also cultured in a similar manner on tissue culture polystyrene (TCP) as a negative control.

SEM of Seeded Scaffolds

To evaluate the morphology of cells growing on the scaffolds, samples were fixed on days 7, 14 and 21 after seeding, as above, and then serially dehydrated in ethanol and hexamethyldisilazane (HMOS) for 10 minutes in each concentration (Raub et al., 2007, Biophys J, 92(6): 2212-22). The samples were left to air dry in a chemical fume hood overnight at room temperature, sputter coated with carbon, and observed under SEM, as described elsewhere herein.

Cell Viability and Proliferation

Cell viability and proliferation over the 21 day period were monitored using the continual alamarBlue™ (AB) assay on days 0, 3, 7, 14 and 21 as previously described (O'Brien et al., 2000, Eur J Biochem, 267(17): 5421-6). In brief, 7F2 cells were seeded in 24 well plates onto TCP, as well as on circular CTS-GP and CTS-HA-GP scaffolds at a density of 3.5×10⁴ cells/well. At the time points stated above, AB was added at 10% (v/v) in triplicate to each well plate and allowed to react in an incubator for three hours. For zero control AB was added also to wells, containing only medium or scaffolds+media. After 3 hours 2000 aliquots of the supernatant were pipetted in triplicate into 96 well plates and the AB fluorescence was read in a Synergy 4 microplate reader (Biotek, Winooski, Vt.) at an excitation wavelength of 545 nm and an emission wavelength of 590 nm. The data were analyzed using Gen5 software (Biotek, Winooski, Vt.) and samples were normalized to their respective zero controls. The cells were re-fed with fresh medium and placed in the incubator to be analyzed at the next time point.

Alkaline Phosphatase (ALP) Assay

ALP activity was measured to assess early osteoblast differentiation towards the osteocytic phenotype (Zhang et al., 2010, Tissue Eng Part A, 16(6): 1949-60). Murine 7F2 osteoblast-like cells were cultured as described above on TCP, CTS-GP and CTS-HA-GP scaffolds in 24 well-plates at a density of 3.5×10⁴ cells/well. The ALP assay was performed on days 0, 7, 14 and 21 using a commercial kit (Abeam, ALP colorimetric assay kit, catalog number ab83369). At each time point, 3 scaffolds were homogenized manually in a glass tube homogenizer containing 300 mL of assay buffer (kit component). The supernatant was collected and centrifuged at 2600 ref for 3 minutes to remove all insoluble debris. 30 μL aliquots of the resultant samples were added to a 96 well plate followed by 50 μL of assay buffer and 50 μL of para-Nitrophenylphosphate (pNPP) solution. Following incubation for one hour at room temperature, 204 of stop buffer was added to the samples and the absorbance was read on the microplate reader at 420 nm. To assess ALP activity of control cells growing on TCP, the wells were rinsed with 300 μL of 1×PBS, followed by addition of 1× lysis buffer for 10 minutes. After that, cell remnants were scraped with a cell scraper for manual lysis and the supernatant was collected. The protocol for analyzing ALP activity was then followed, as discussed elsewhere herein.

RNA Isolation and Real Time RT-PCR

7F2 cells were trypsinized after 24 hours, 2 week, and 3 week of culture on TCP, CTS-GP and CTS-HA-GP scaffolds. The cells were pelleted by centrifugation at 800 RPM for 5 minutes. After a 1×PBS wash, the resulting pellet was stored at −80° C. prior to RNA isolation. A Qiagen RNeasy Mini Kit was used to isolate RNA by columnar centrifugation and DNase digestion, as per the manufacturer's instruction. RNA integrity was initially determined by electrophoresis on 1.0% agarose gels. Real-time polymerase chain reaction (RT-PCR) was performed with a Qiagen One-Step Kit combined with TaqMan expression assays according to the manufacturer's instructions. Quantitative RT-PCR was performed using a Realplex II real-time PCR machine (Eppendorf, Hamburg, Germany) using the following Taqman primers (from Applied Biosystems, Carlsbad, Calif.): Spook (Osteonectin, Mm00486393_m1), Alkaline Phosphatase (Mm01187115_m1), Sppl (Osteopontin, Mm00436767_m GAPDH (Hs99999905_ml) was used as an internal ‘housekeeping’ control.

Primer efficiency was determined by linear regression of a dilution series, CT results were analyzed by the Pfaffl Method and the results were normalized to TCP and GAPDH prior to logarithmic transformation (Hunt, 2010, Microbiology and Immunology Online, avail from: http://pathmicro.med.sc.edu/pcr/realtime-home.htm). Each experimental condition and gene primer was analyzed in triplicate.

Statistical Analysis

Unless stated otherwise, all experiments were repeated at least three times in triplicate: All data are presented as mean value±a standard deviation. Results were analyzed using a one-way ANOVA test of variance with an ad hoc Tukey Test; Results with p-values of <0.05 (*) and, 0.01 (**) were considered statistically significant. The results of the experiments are now described.

### Morphological Characterization of Genipin Crosslinked Chitosan/Hydroxyapatite Nanofibers

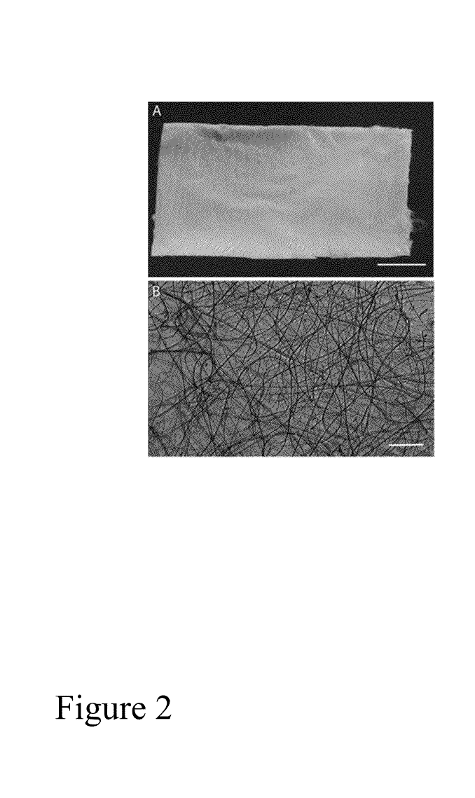

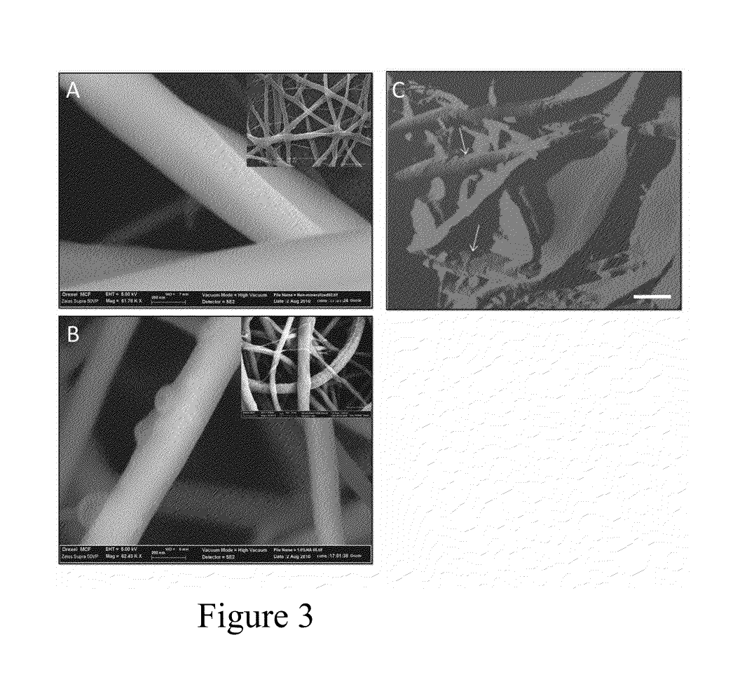

Optimization of the electrospinning process for obtaining pure chitosan (CTS) and chitosan-hydroxyapatite (CTS-HA) fibers was required because to date, no one has electrospun CTS-HA nanofibers without the use of a fiber-forming high molecular weight additives, such as poly(ethylene oxide) (PEO). The optimization process included first systematically adjusting the concentration, flow rate, working distance and voltage of the electrospinning set up, as described before (Li et al., 2002, J Biomed Mater Res, 60(4): 613-21; Li et al., 2006, Biomaterials, 27(13): 2705-15; Han et al., 2010, Biomacromolecules; Li et al., 2005 Conf Proc IEEE Eng Med Biol Sec, 6: 5858-61), yielding electrospun fibers were continuous and uniform in shape, without beading (FIG. 2B). Once bead-less fibers were obtained, final parametric adjustments were made until smaller scale nanofibers were obtained. The thickness of a typical, optimized, electrospun nanofibrous scaffold mat (FIG. 2A), as evaluated by Equation 1, was 25.3±16.2 μm. The diameters of the individual non-crosslinked nanofibers fibers in the mat, evaluated by SEM, were 227.8±154.3 nm, demonstrating a rather wide large heterogeneity. In contrast to the smooth “native” CTS-GP fibers (FIG. 3A), CTS-HA-GP fibers (FIG. 3B) contained nanoparticles with an average height of 42.4±11 nm, as determined by AFM analysis (FIG. 3C). After hydration, the morphology of the fibers changed to a flatter shape for the CTS-GP nanofibers, but retained a more rounded shape for the CTS-HA-GP nanofibers (see inserts in FIGS. 3A and 3B). Crosslinking and hydration caused an increase in the diameter of the fibers to 334.7±119.1 nm, which was not significantly different from that of the non-crosslinked fibers (one-way ANOVA with Tukey Test, p<0.05).

### Evaluation of Nanoparticle Deposits on CTS-HA-GP Nanofibers

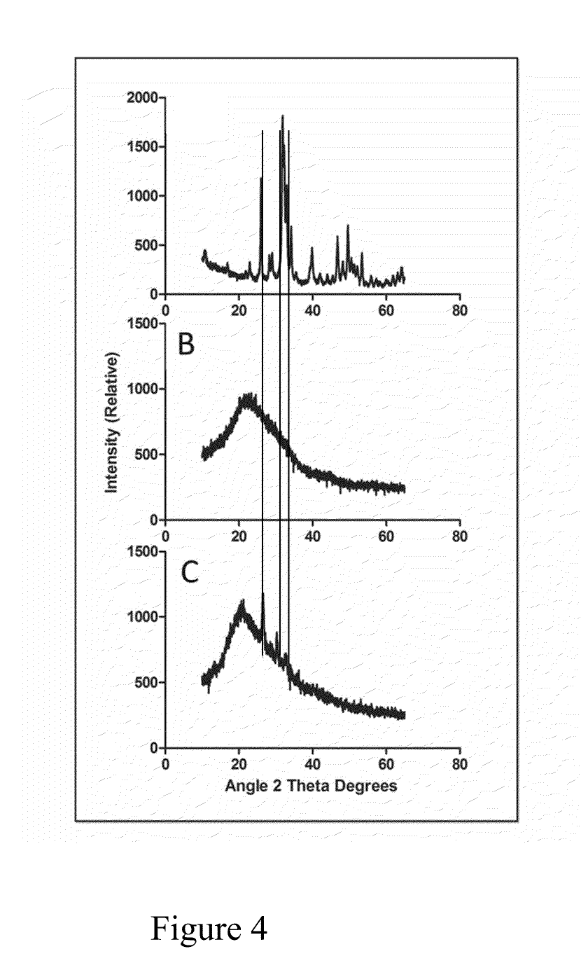

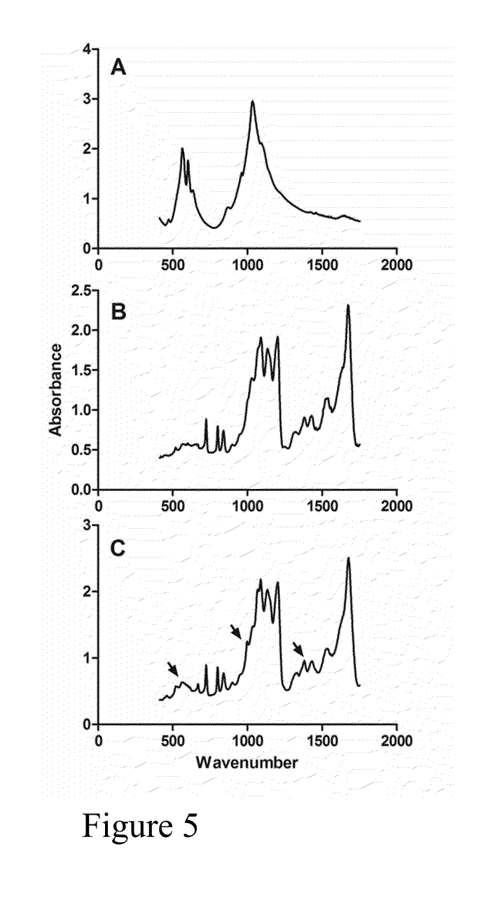

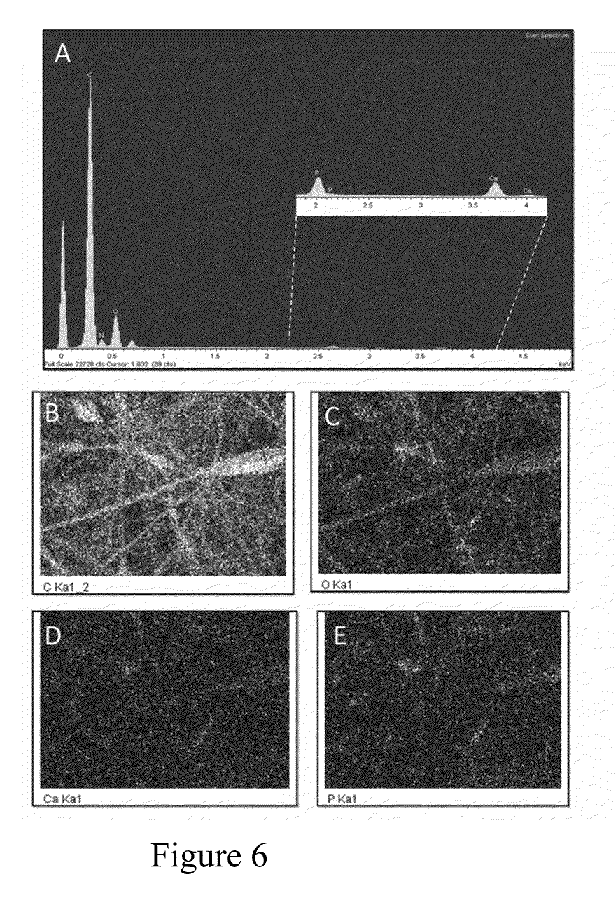

Three independent approaches, XRD, FTIR and EDS, were employed to further characterize the nanoparticle deposits observed on the surface of the CTS-HA-GP nanofibers (FIG. 3B). XRD spectroscopy revealed a characteristic, highly crystalline structure for the pure HA powder (FIG. 4A), while the spectrum of the non-mineralized CTS-GP scaffolds resembled that of amorphous electrospun CTS (FIG. 4B). The spectra for the CTS-HA-GP composite fibers containing nanoparticles showed three distinct HA peaks at 26.21, 30.24 and 32.41 degrees, indicating the incorporation of HA crystalline properties into the amorphous nanostructure of the CTS-GP scaffolds (FIG. 4C). The phosphate groups in pure HA show characteristic FTIR bands between 1000-1100 cm⁻¹ and 500-600 cm⁻¹ (FIG. 5A) (Kumirska et al., 2010, Mar Drugs, 8(5): 1567-636). FTIR Spectra of the CTS-HA-GP biocomposites reveal bands at 500-600 cm⁻¹ (FIG. 5C) that do not appear in the CTS-GP non-mineralized scaffold spectra (FIG. 5B). Additionally, there is a broadening of the band around 1050 cm⁻¹ that has been attributed to the interaction of HA and chitosan (FIG. 5C) (Kumirska et al, 2010, Mar Drugs, 8(5): 1567-636). Finally, EDS was used to determine the elemental composition of the individual nanofibers. CTS-GP scaffolds showed large peaks for carbon and oxygen and a small peak for nitrogen, indicating three of the main components of the organic material (FIG. 6A). In fibers containing 1.0% HA, small amounts of calcium and phosphorus were found (insert in FIG. 6A). Concentrations of 0.8% HA yielded still smaller peaks, while concentrations of 2.0% HA yielded peaks similar to 1.0% suggesting a saturation of HA that can be incorporated into these fibers. Dot mapping spectral analysis of EDS showed the distribution of carbon (FIG. 6B) and oxygen (FIG. 6C) as the organic components, and calcium (FIG. 6D) and phosphate (FIG. 6E) in the form of HA nanoparticles incorporated in/on the fibers.

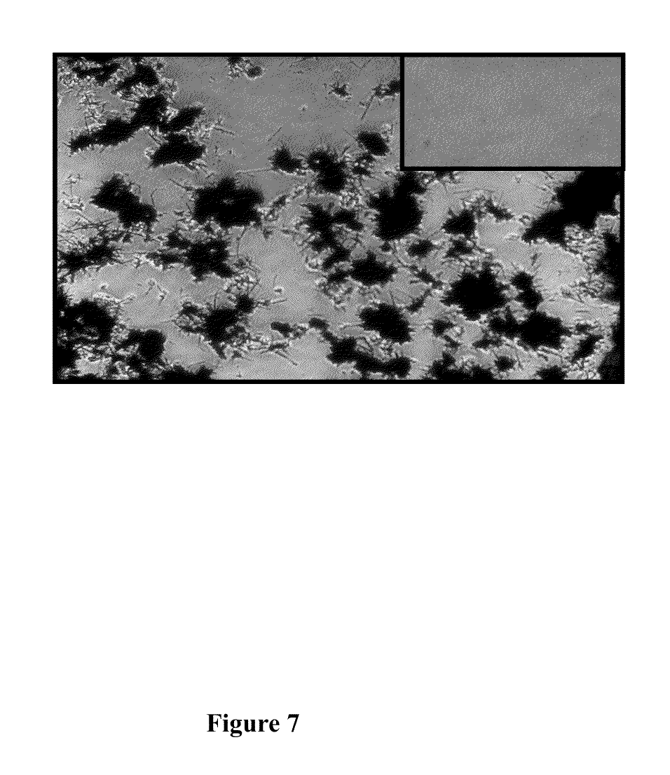

Von Kossa staining of 0.1% genipin crosslinked 1.0% mineralized 7% chitosan nanofibers demonstrated the presence of micro-sized calcium deposits on the surface of the scaffold (FIG. 7).

### Mechanical Properties of CTS-HA-GP Nanofibers

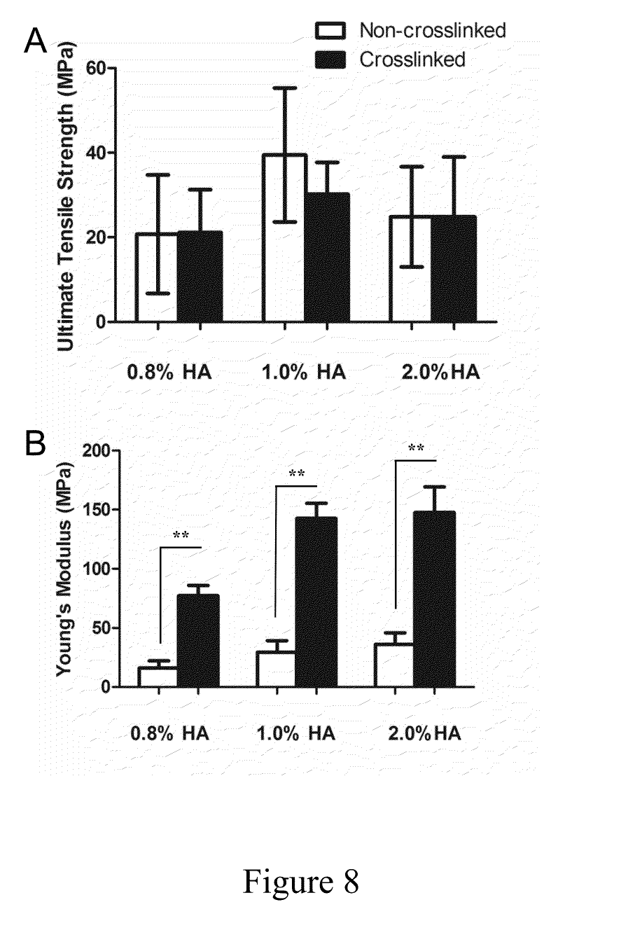

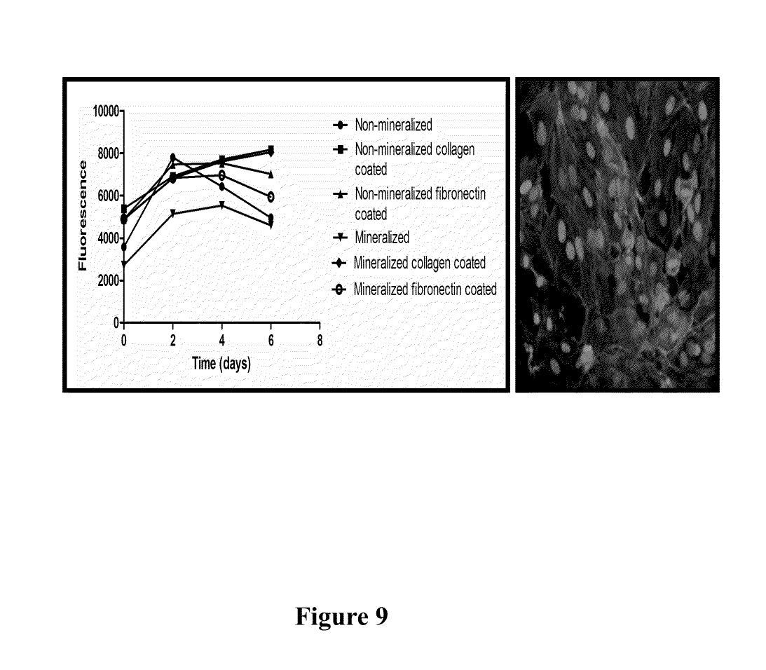

Three different concentrations of HA were used to evaluate the effect of mineralization on the mechanical properties of genipin crosslinked scaffold. As shown in FIG. 8 the ultimate tensile strength (UTS), as determined by the stress at break as a function of the cross-sectional area of the scaffold, shows no significant difference by increasing the concentration of HA from 0.8%-2.0%; there was also no significant difference due to GP crosslinking (FIG. 8A). Increasing the HA contents from 0.8%, 1.0%, and 2.0% % did not significantly increase the Young's moduli of non-crosslinked CTS-HA scaffolds. By contrast, raising the HA contents significantly increased the Young's moduli of crosslinked CTS-HA-GP samples from 79.5±8.1 to 124.2±21.8 and 130.1±24.8 MPa for 0.8%, 1.0% and 2.0% HA respectively, Crosslinking with genipin caused a significant 4-5 fold increase (p<0.01) in stiffness for all samples (FIG. 8B).

### Morphology of 7F2 Osteoblasts on CTS-HA-GP Scaffolds

The cellular biocompatibility of the chitosan nanofibers was assessed by evaluating the capability of osteoblasts to proliferate on different conditions of 0.1% genipin crosslinked non-mineralized and 1.0% mineralized 7% chitosan nanofibers and various coated chitosan nanofibers. Cell growth was monitored with alamarBlue™ (AB) assay for up to one week (FIG. 6). It was observed that osteoblasts adhered and proliferated on the scaffolds for 7 days independent of the mineralization or extracellular matrix protein coating.

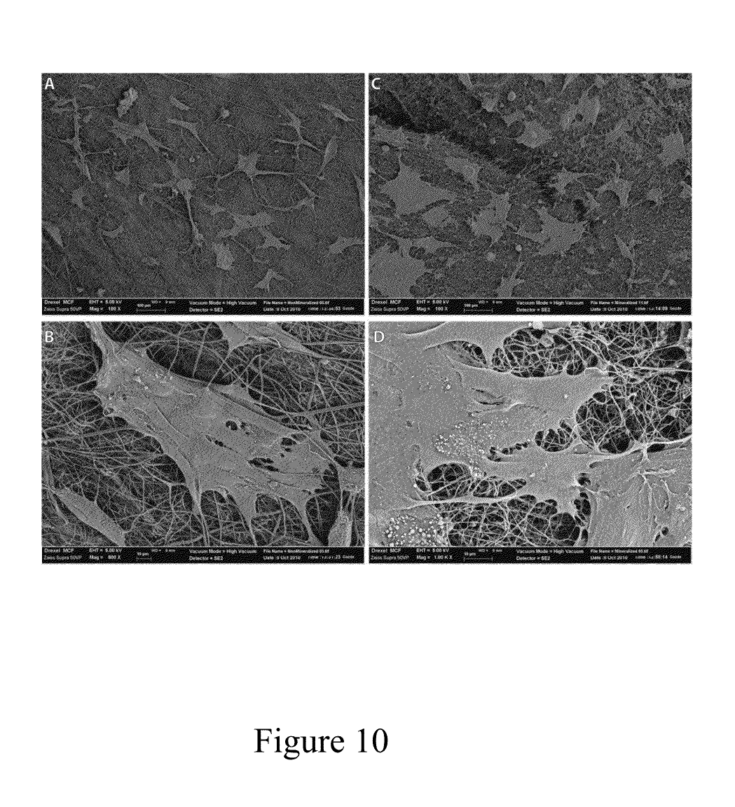

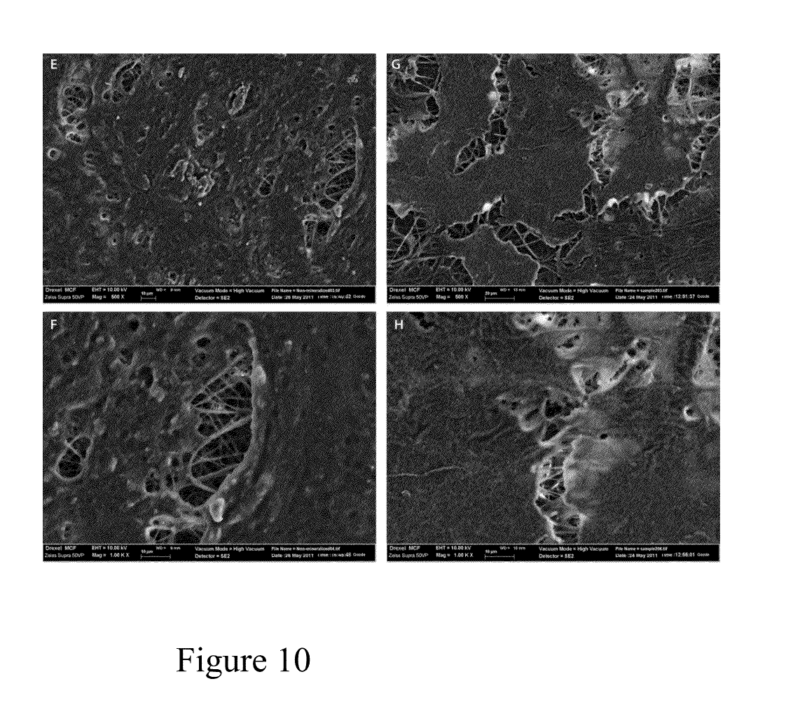

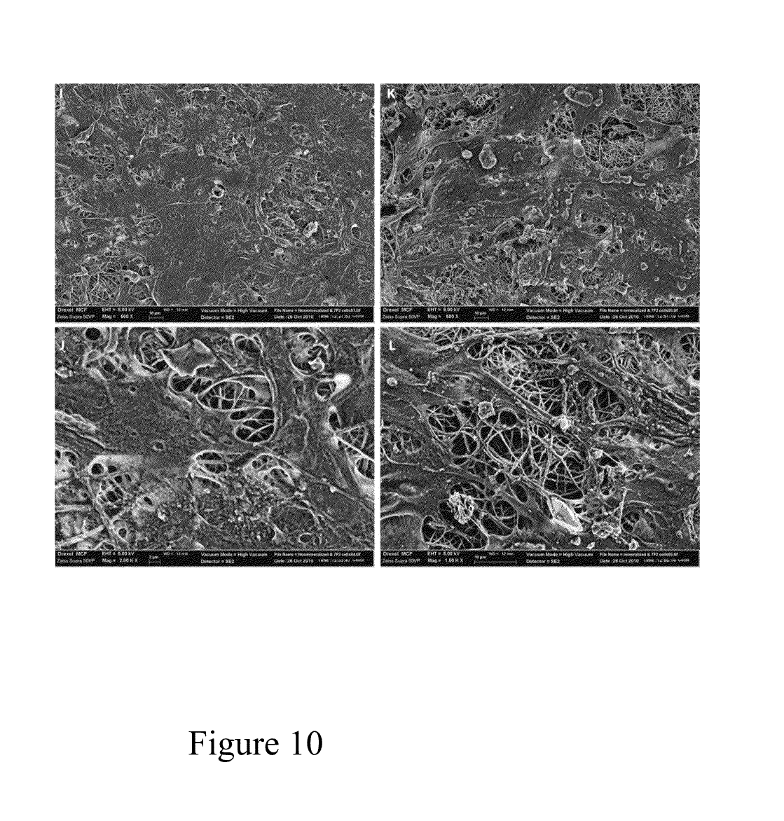

The morphology of the cells cultured on CTS-GP and CTS-HA-GP nanofibers was evaluated by SEM. On day 7 following seeding, cells formed extensive cell-scaffold and cell-cell interactions as inferred from well-defined fillopodia extending from the lamellipodia and “grabbing” the nanofibers on both non-mineralized and composite scaffolds indicative of cellular proliferation/migration and cell-cell and cell-scaffold communication (FIGS. 10A, 10B, 10C and 10D). By day 14, the cells appear well spread on both the CTS-GP and the biocomposite scaffolds (FIGS. 10E, 10F, 10G and 10H) and maintain this configuration at least through day 21 (FIGS. 10I, 10J, 10K and 10L).

### Capacity of CTS-HA-GP Scaffolds to Induce Osteogenic Differentiation In Vitro

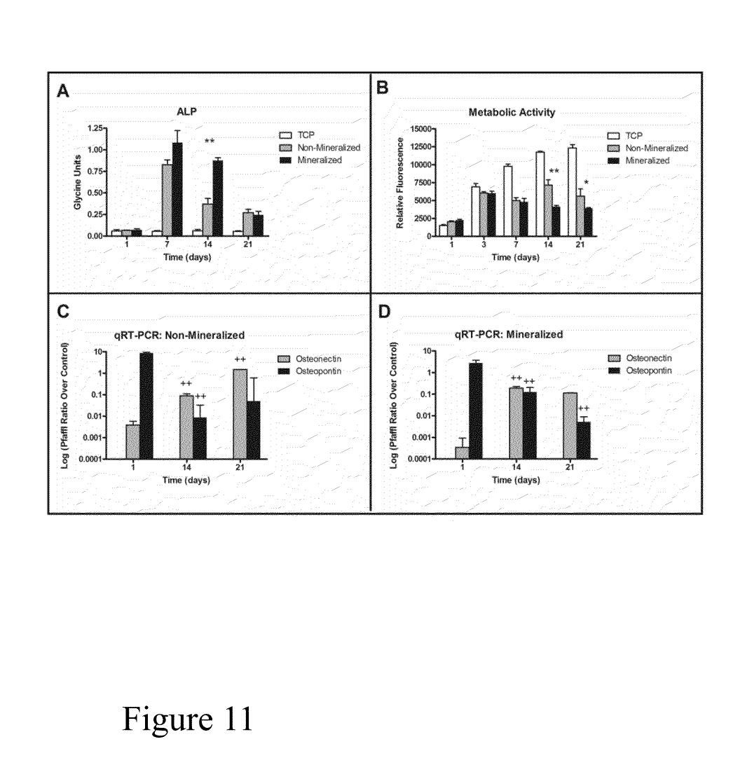

7F2 cells, deemed pre-osteoblast-like cells, secrete alkaline phosphatase (ALP), and upon differentiation mineralize their own matrix just like mature osteocytes (Saad et al., 2011, Int Orthop, 35(3): 447-51). The osteoinductive potential of the scaffolds was assessed using several independent approaches. The activity of ALP, an early osteogenic marker, was evaluated using a colorimetric pNPP assay on days 7, 14, and 21 post seeding (FIG. 11A). When grown on tissue culture plastic (TCP,) 7F2 cells consistently had lower ALP activity than when grown on the cross-linked CTS scaffolds (p<0.01). Furthermore, by day 14, ALP activity in cells growing on the mineralized, biocomposite scaffolds was 2.4 fold higher than on non-mineralized scaffolds, (p<0.01). Expression of this early osteogenic marker decreased on both scaffolds by day 21 as differentiation continued and cells matured. Cell metabolic activity was assessed continually using the alamarBlue™ (AB) assay. In the first 3 days following seeding, the metabolic activity increased in all samples. On TCP, AB fluorescence continued to increase over 21 days while it remained stable on non-mineralized samples and decreased in mineralized samples (FIG. 11B). Cells cultured on TCP had the highest AB fluorescence at all time points, indicating maximal metabolic activity and may also indicate a minimal amount of differentiation. For cells on CTS-HA scaffolds AB fluorescence remained relatively stable over the time course of the experiments after the first initial increase, suggesting a decreased metabolic activity compared to TCP, which may indicate the cells are beginning to undergo differentiation. AB fluorescence on CTS-HA-GP composite nanofibers decreases over time, which may indicate enhanced differentiation in comparison to the CTS-HA scaffolds.

Early and late markers of osteogenic differentiation were also monitored by qRT-PCR, measuring RNA expression of osteopontin (OP) and osteonectin (ON), respectively. As seen in FIGS. 11C and 11D, the expression of OP, an early marker of osteogenic differentiation is highest 24 hours after cell seeding and then decreases progressively at days 14 and 21 on both the non-mineralized and the mineralized composite scaffolds. The expression of ON mRNA, which is a late marker of osteogenic maturation, is at its lowest levels at 24 hours and increases on days 14 and 21 in cells growing on the non-mineralized samples. However, on the mineralized samples, a significantly larger increase (by ˜2 orders of magnitude) in ON expression between 24 hours and day 14 was observed (with a subsequent plateau at day 21), compared to that on the non-mineralized scaffolds, indicating that the presence of HA accelerates/enhances osteogenic differentiation/maturation of 7F2 cells.

As described herein, a fibrous scaffold was engineered to architecturally resemble bone matrix, while possessing mechanical properties of periosteum. Past studies described two-step processes for the formation of mineralized fibers, in which the incorporation of HA into electrospun nanofibers was achieved by synthesizing a co-precipitation solution consisting of HA and the polymer of interest (Zhang et al, 2010, Tissue Rug Part A, 16(6): 1949-60; Catledge et al, 2007, Biomed Mater, 2(2): 142-50). However, as described herein, a simpler, one-step solution is disclosed by co-dissolving CTS and HA in TFA and allowing ample stirring to create a homogenous, electrospinnable solution. Furthermore, by increasing the concentration of chitosan from 23% to 7%, the thickness of the scaffolds is increased by >2 fold, from 10.1±5.8 μm (Schiffman et al., 2007, Biomacromolecules, 8(2): 594-601) to 25.3±16.2 μm. Additionally, as described herein, CTS nanofibers were electrospun without the use of a fiber-forming aid, such as ultrahigh molecular weight polyethylene oxide UHMWPEO (Zhang et al, 2010, Tissue Eng Part A, 16(6): 1949-60), which is often used to enhance chain entanglement of materials that do not have high electrospinnability (Zhang et al., 2008, Biomacromolecules, 9(1): 136-41).

To evaluate the topography, structure, fiber composition, and HA incorporation in the electrospun scaffolds, 5 independent materials characterization techniques, SEM, AFM, XRD, FTIR, and EDS were used. Analysis of SEM micrographs (FIG. 3), revealed a mean fiber diameter of 227.8±154.3 nm. This large variability/heterogeneity in the size of electrospun CTS nanofibers is commonly seen in the recent literature (Xie et al., 2010, Biomed Mater, 5(6): 065016; Zhang et al, 2010, J Biomed Mater Res A, 95(3): 870-81; Cai et al, 2010, Int J Mol Sci 11(9): 3529-39). Without being bound to any particular theory, this may be due to inhomogeneity of the solution. Chitosan has an extremely high surface tension, which is why TFA is required as a solvent (Schiffman et al., 2007, Biomacromolecules, 8(2): 594-601). No other organic solvents or weak acids have been able to overcome the surface tension to form uniform fibers. Hence the large variability in fiber diameter may be caused by the harsh conditions required to make chitosan electrospinnable. The nano-scale of the HA particles (42.4±11.0 nm) incorporated on the surface of CTS-HA-GP composite fibers was measured by AFM, which also showed the rough topography of the mineralized scaffolds (FIG. 3C).

By using EDS, it was found that the 1% HA containing mineralized nanofibers contained small amounts of calcium and phosphorous. Analysis of the peak heights indicated that the biocomposite scaffold was composed of approximately 14.3% HA. The EDS peaks seen in FIG. 6A show the presence of the main organic components of the chitosan: carbon, oxygen and nitrogen, and smaller phosphorous and calcium peaks are present in addition on the mineralized CTS-GP scaffolds (insert). To confirm uniformity of the nanoparticle distribution, dot map analysis was performed. Carbon and oxygen (FIGS. 6B and 6C), represented as white dots, make up the main components of the nanofibers while calcium and phosphorous (FIGS. 6D and 6E) are dispersed evenly and in parallel with the carbon and oxygen. Heinemann et al. (Heinemann et al., 2008, Biomacromolecules 9(10): 2913-20) discussed the use of energy dispersive x-ray mapping (EDX), synonymous with EDS, in order to qualitatively discuss the presence of calcium and phosphorous on collagen coated chitosan scaffolds after seeding with 7F2 cells. Post-seeding, Ca and P peaks were seen present on EDX maps, which were deemed indicative of the presence of HA due to the mineralization of the scaffolds from matrix deposition of the 7F2 cells (Danilchenko et al., 2011, J Biomed Mater Res A, 96(4): 639-47).

FTIR was used to quantify the molecular interactions and vibrations present in the chitosan scaffolds. While calcium is vibrationally “undetectable”, some small bands between 1000-1100 cm⁻¹ and 500-600 cm⁻¹ were noted on the mineralized scaffold (FIG. 5). Without being held to any particular theory, the bands might correspond to PO₄ in HA (Kumirska et al, 2010, Mar Drugs, 8(5): 1567-636; Danilchenko et al., 2011, J Biomed Mater Res A, 96(4): 639-47). Broadening of the peak at 1050 cm⁻¹ and superposition of 1550-1700 cm⁻¹ peaks (see FIG. 5) have been attributed to the interaction of HA and chitosan (Danilchenko et al., 2011, J Biomed Mater Res A, 96(4): 639-47). As additional evidence for HA incorporation, the XRD spectra of the biocomposite mineralized scaffolds show defined peaks specifically matching those found in pure HA samples (FIG. 4). The peaks in the CTS-HA-GP spectra are an indication of semi-crystalline structures present in the composites as opposed to the completely amorphous nature of CTS-GP scaffolds. Analysis of the 3 indicative peaks in the biocomposite scaffold relate to approximately 22.6%, 7.8% and 4.0% from left to right to the compared peaks in the pure HA sample (FIG. 4).

A prevalent concern with engineered tissues is to chemically and structurally mimic the native tissue, and to approximate its unique mechanical properties. While electrospun scaffolds morphologically resemble the fibrous structure of the ECM, they are generally thin and mechanically inferior to scaffolds prepared by some other fabrication techniques, such as lyophilization. Crosslinking can help to enhance the mechanical properties and fine tune them to the desired strength. As described herein, OP was used as a natural, non-toxic crosslinker (Mang et al., 2010, J Biomed Mater Res A, 95(3): 870-81; Bispo et al., 2010, Nanotechnol, 6(2), 166-75; Solorio et al., 2010, Tissue Eng Regen Med, 4(7): 514-23). Without being held to any particular theory, it is proposed that, upon crosslinking with GP, the fibers form new intramolecular bonds between each other. This in turn causes an increase in the scaffold stiffness, as observed by a significant increase in the Young's modulus in the absence of a significant increase in the ultimate tensile strength (FIGS. 8A and B). Recently Zhang et al reported that incorporation of HA reduces the mechanical strength of electrospun chitosan/collagen scaffolds (Zhang et al., 2010, Tissue Eng Part A, 16(6): 1949-60). The Young's modulus of their non-crosslinked chitosan/UHMWPEO scaffolds was 92.2±19.1 MPa and decreased to 57.3±15.5 MPa and 48.2±8.3 MPa upon incorporation of HA and HA/collagen respectively. The Youngs' modulus of the 1.0% mineralized non-crosslinked, waterproofed chitosan scaffolds described herein was 25.2±9.2 MPa (FIG. 8B). Without being bound to any particular theory, the lower values for the scaffolds described herein may reflect the absence of UHMWPEO or, alternatively be due to the testing method used herein. The scaffolds described herein were tested while wet in order to closely mimic the natural tissue. The mechanical properties of the dry scaffolds are not applicable once the scaffolds are placed into contact with the aqueous environment of the human body. Upon crosslinking with GP the Young's modulus of the scaffolds described herein increased about 5 fold to 124±21.8 MPa, which is comparable to the stiffness of porcine periosteum, which is comparable to human, that reportedly ranges from about 60-100 MPa depending on the location and the load bearing properties of the bone (Popowics et al., 2002, Arch Oral Biol, 47(10): 733-41).

Periosteum plays a central role in the health of bone tissue. The periosteum is the source and site for the recruitment of osteoprogenitor cells that are responsible for new bone formation at sites of injury, as highlighted by studies comparing the effects of ablating different sources of osteoprogenitor cells. While removing such progenitor cells from the bone marrow had minimal effect, removal of periosteum caused a 73% decrease in new bone formation (Zhang et al., 2005, J Bone Miner Res, 20(12): 2124-37; Tiyapatanaputi et al., 2004, J Orthop Res, 22(6): 1254-60). The osteogenic properties of the mineralized and non-mineralized CTS-GP fibrous scaffolds were assessed using 7F2 mouse osteoblast like cells. As seen in FIG. 11B, the metabolic activity of the cells (as inferred from AB fluorescence) decreased over time in cells cultured on HA-containing scaffolds. When cells undergo differentiation, they cease proliferating, which may be indicated by a decrease in metabolic activity. Hence, the decrease in AB fluorescence may be due to an increase in differentiation of the cells, which corresponds to the increase in ALP activity seen in FIG. 11A. Based on these data and the SEM images shown in FIGS. 10A-L it is surmised that the metabolic activity plateaued before the cells reached confluency on either scaffolds, due to the cells beginning to undergo differentiation from the mechanical cues of the scaffold.

In line with previous studies (Zhang et al., 2008, Biomaterials 29(32): 4314-22; Zhang et al., 2010, Tissue Eng Part A, 16(6): 1949-60), ALP activity was promoted significantly (p<0.05) by the mineralized biocomposite scaffolds at days 7 and 14 as compared to both non-mineralized and TCP. Data described herein suggests that both surface topography of the substrate and innate biochemical cues in the scaffolds play important roles in the osteogenic maturation process. PCR data for OP and ON (FIGS. 11C and D) suggests that both CTS-GP and CTS-HA-GP scaffolds promote 7F2 maturation/differentiation however, the cells cultured on the mineralized scaffolds matured at a faster rate, as inferred from the sharp increase from day 0 to day 14 in ON mRNA expression on CTS-HA-GP scaffolds as compared to the more gradual and linear increase on CTS-GP scaffolds (p<0.01).

As described herein, a simple, one-step technology is disclosed, wherein the technology is used to generate electrospun and mineralized fibrous chitosan scaffolds that are subsequently crosslinked with genipin as potential substitutes for periosteum. Crosslinking with genipin resulted in five-fold increase in mechanical properties, approximating those of periosteum. Osteoinductive bioactivity of the scaffolds was tested in vitro using 7F2 osteoblast-like cells. Based the results disclosed herein, it is proposed that electrospun crosslinked mineralized chitosan nanofibrous scaffolds are good candidates for non-weight bearing bone tissue engineering. Without wishing to be bound by any particular theory, it is believed that the scaffolds of the invention can induce osteogenic differentiation in human bone marrow derived mesenchymal stem cells as well as repair craniofacial lesions.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A scaffold comprising an electroprocessed cross-linked mineralized chitosan nanofiber, wherein the scaffold is substantially free of a fiber forming agent and wherein the scaffold is capable of supporting the maturation of an osteoblast.

2. The scaffold of claim 1, wherein the mineralized chitosan nanofiber comprises hydroxyapatite.

3. The scaffold of claim 1, wherein the mineralized chitosan fiber is cross-linked with genipin.

4. The scaffold of claim 1, wherein the fiber forming agent is polyethylene oxide.

5. The scaffold claim 1, wherein the scaffold exhibits mechanical properties of natural bone.

6. The scaffold claim 1, wherein the scaffold further comprises a cell selected from the group consisting of an osteoprogenitor cell, a mesenchymal cell, a stem cell, an osteoblast, an osteocyte, and any combination thereof.

7. A method of making a scaffold comprising an electroprocessed cross-linked mineralized chitosan nanofiber, the method comprising:
(a) dissolving chitosan and hydroxyapatite in an acidic solution to form a first solution,
(b) electroprocessing the first solution to form a fibrous mat, and
(c) cross-linking the fibrous mat with genipin to form a scaffold comprising an electroprocessed cross-linked mineralized chitosan nanofiber.

8. The method of claim 7, wherein the acidic solution comprises trifluoracetic acid.

9. The method of claim 7, further comprising contacting a cell with the scaffold, wherein the cell is selected from the group consisting of an osteoprogenitor cell, a mesenchymal cell, a stem cell, an osteoblast, an osteocyte, and any combination thereof.

10. A method of treating a bone defect in a mammal, the method comprising administrating to the site of a bone defect a scaffold comprising an electroprocessed cross-linked mineralized chitosan nanofiber, wherein the scaffold is substantially free of a fiber forming agent and wherein the scaffold is capable of supporting the maturation of an osteoblast.

11. The method of claim 10, wherein the mineralized chitosan nanofiber comprises hydroxyapatite.

12. The method of claim 10, wherein the mineralized chitosan fiber is cross-linked with genipin.

13. The method of claim 10, wherein the fiber forming agent is polyethylene oxide.

14. The method claim 10, wherein the scaffold exhibits mechanical properties of natural bone.

15. The method claim 10, wherein the scaffold further comprises a cell selected from the group consisting of an osteoprogenitor cell, a mesenchymal cell, a stem cell, an osteoblast, an osteocyte, and any combination thereof.
